(19) United States
(12) Patent Application Publication
(10) Pub. No.: US 2004/0266688 A1
(43) Pub. Date: Dec. 30, 2004

(54) Methods for modulating endometrium

(76) Inventor: Nihar R. Nayak, Mountain View, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/846,477

(22) Filed: May 14, 2004

Related U.S. Application Data
(60) Provisional application No. 60/470,683, filed on May 14, 2003.

(51) Int. Cl. A61K 38/17

(52) U.S. Cl. 514/12

(57) ABSTRACT

The present invention provides methods of modulating the endometrium, including methods for inhibiting pregnancy and methods for treating gynecological bleeding disorders, such as dysfunctional uterine bleeding and endometrial bleeding. The methods generally involve administering to an individual in need thereof a vascular endothelial growth factor (VEGF) antagonist, e.g., a chimeric VEGF blocker, where the VEGF antagonist is administered during the post-menstrual repair phase. The invention further provides chimeric VEGF blockers formulated for use in inhibiting pregnancy and treating gynecological bleeding disorders, as well as kits and delivery systems comprising the formulations.

### CROSS-REFERENCE

This application claims the benefit of U.S. Provisional Patent Application No. 60/470,683 filed May 14, 2003, which application is incorporated herein by reference in its entirety.

### FIELD OF THE INVENTION

The present invention is in the field of regulation of the endometrium, e.g., postmenstrual healing and regeneration of primate endometrium, and in particular in methods of regulating fertility and methods of treating gynecological bleeding disorders.

### BACKGROUND OF THE INVENTION

The vast majority of oral contraceptives consists of a combination of a progestin and estrogen that are administered concurrently for 21 days followed either by a 7 day pill free interval or by the administration of a placebo for 7 days in each 28 day cycle. Oral contraceptives currently in use include Norgestimate, Norethindrone, Norgestrel, Desogestrel, Norethindrone acetate, and Levonorgestrel.

Use of oral contraceptives is associated with side effects, including stroke, ischemic heart disease, thromboembolic disorders, and endometrial bleeding. Despite efforts to develop new forms of birth control that avoid these side effects, use of currently available oral contraceptives is still associated with risks.

Endometrial bleeding is associated with the use of progestogen-only contraceptives. Endometrial bleeding that is induced by the use of progestogens can be treated, but current treatments are of limited effectiveness. Thus there is considerable interest in finding both a treatment that works and a way to avoid the bleeding in the first place.

Dysfunctional uterine bleeding is the most common cause of abnormal vaginal bleeding during a woman's reproductive years. The diagnosis of dysfunctional bleeding may be made when other organic and structural causes for abnormal vaginal bleeding have been ruled out. A normal menstrual cycle occurs every 21-35 days with menstruation for 2-7 days. The average blood loss is 35-150 ml. Menorrhagia is the term for excessive bleeding with a normal interval. Metrorrhagia is bleeding that is irregular or too frequent. Menometrorrhagia is excessive bleeding at irregular intervals.

As many as 10% of women in the United States with normal ovulatory cycles reportedly have experienced dysfunctional uterine bleeding. Dysfunctional uterine bleeding occurs most frequently in adolescents, e.g., shortly after the onset of menstruation, and in premenopausal women.

Current treatments for dysfunctional uterine bleeding include gestagens, large dose estrogen/gestagen combinations, and nonsteroidal cyclooxygenase inhibitors. However, in view of the known risks associated with currently available treatments, there continues to be a need in the art for new treatments for this disorder. The present invention addresses this need.

Literature

U.S. Pat. No. 6,441,027; U.S. Pat. No. 6,011,003; U.S. Pat. No. 6,100,071; Kressin et al. (2001) Fertil. Steril. 76:1220-1224; Brenner et al. (2002) Ann. N.Y. Acad. Sci. 955:60-74; Tan et al. (2002) Fertil. Steril. 78:148-153; Garrido et al. (2001) Fertil. Steril. 76:568-575; Nayak and Brenner (2002) J. Clin. Endocrinol. Metab. 87:1845-1855; Hastings et al. (2003) Endocrinol. 144:326-334; Holash et al. (2002) Proc. Natl. Acad. Sci. USA 99:11393-11398; WO 02/60489; U.S. Pat. No. 5,712,380; WO 97/44453; WO 97/13787.

### SUMMARY OF THE INVENTION

The present invention provides methods of modulating the endomefrium, including methods for inhibiting pregnancy and methods for treating gynecological bleeding disorders, such as dysfunctional uterine bleeding and endometrial bleeding. The methods generally involve administering to an individual in need thereof a vascular endothelial growth factor (VEGF) antagonist, e.g., a chimeric VEGF blocker, where the VEGF antagonist is administered during the post-menstrual repair phase. The invention further provides chimeric VEGF blockers formulated for use in inhibiting pregnancy and treating gynecological bleeding disorders, as well as kits and delivery systems comprising the formulations.

### BRIEF DESCRIPTION OF THE DRAWINGS

FIGS. 1A-D depict inhibition of the mid-proliferative vascular development and reepithelialization of the rhesus macaque endometrium by blockade of VEGF action during the postmenstrual repair phase by VEGF-trap.

FIG. 2 is a schematic representation of a VEGF blocker suitable for use in a subject method.

FIGS. 3A-C depict various amino acid sequences for inclusion in a suitable VEGF blocker.

FIG. 4 depicts the amino acid sequence of an exemplary chimeric VEGF blocker (SEQ ID NO:16).

### DEFINITIONS

As used herein, the terms “treatment,” “treating,” and the like, refer to obtaining a desired pharmacologic and/or physiologic effect. The effect may be prophylactic in terms of completely or partially preventing a condition, disease, or symptom thereof and/or may be therapeutic in terms of a partial or complete cure for a disease or condition, and/or adverse or undesired effect attributable to the disease or condition. “Treatment,” as used herein, covers any treatment of a disease in a mammal, particularly in a human, and includes: (a) preventing the disease or condition from occurring in a subject which may be predisposed to or at risk of developing the disease or condition but has not yet been diagnosed as having it; (b) inhibiting the disease or condition, i.e., arresting its development; and (c) relieving the disease or condition, e.g., causing regression of the disease or condition, e.g., to completely or partially remove symptoms of the disease or condition.

As used herein, the term “contraception” refers to an inhibition of pregnancy, and includes contragestation, e.g., inhibition of implantation of a blastocyst into the endometrium of the uterus. The term “contraception” is used interchangeably herein with the terms “regulating fertility,” and “inhibiting pregnancy,” and grammatical equivalents of such terms.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, vaginal swabs, vaginal fluids, cell supernatants, cell lysates, serum, plasma, biological fluids, and tissue samples.

The term “gynecological bleeding disorder,” as used herein, refers to any of a variety of gynecological bleeding disorders, including, but not limited to, dysfunctional menstrual bleeding, menorrhagia, metrorrhagia, menometrorrhagia, endometrial bleeding, and the like.

As used herein, the term “vascular endothelial growth factor antagonist,” or “VEGF antagonist” refers to any agent that antagonizes an effect of VEGF, including an agent that blocks the binding of VEGF to a VEGF receptor (e.g., VEGF-R1 or VEGF-R2); and an agent that inhibits the transduction of a signal mediated by a VEGF receptor, e.g., a signal that is mediated by binding of a ligand or other VEGF receptor binding agent to a VEGF receptor.

The term “chimeric VEGF blocker” as used herein refers only to structure and is not limiting as to the method of production, source (synthetic, recombinant, etc.), or source of component parts (e.g., species, receptor subtypes, etc.).

The terms “individual,” “subject,” “host,” and “patient,” used interchangeably herein, refer to a mammal, particularly to a primate, e.g., a human, particularly a female human.

As used herein, the terms “determining” and “detecting” refer to both quantitative and qualitative determinations and as such, the terms “determining” and “detecting” are used interchangeably herein with “assaying,” “measuring,” and the like.

The term “dosing event” as used herein refers to administration of a therapeutic agent (e.g., a VEGF antagonist, etc.) to a patient in need thereof, which event may encompass one or more releases of agent from a drug dispensing device.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a soluble VEGF receptor” includes a plurality of such receptors, reference to “a VEGF antagonist” includes a plurality of such antagonists, and reference to “the formulation” includes reference to one or more formulations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of modulating the endometrium, which methods can include methods for inhibiting pregnancy (e.g., inhibiting implantation of a blastocyst into the uterine wall), and methods for treating gynecological disorders resulting from abnormal endometrial vascular development. The methods generally involve administering to an individual a vascular endothelial growth factor (VEGF) antagonist to a female human during the postmenstrual repair phase. The invention further provides chimeric VEGF blockers formulated for use in inhibiting pregnancy (e.g., inhibiting implantation) and in treating gynecological disorders such as gynecological bleeding disorders.

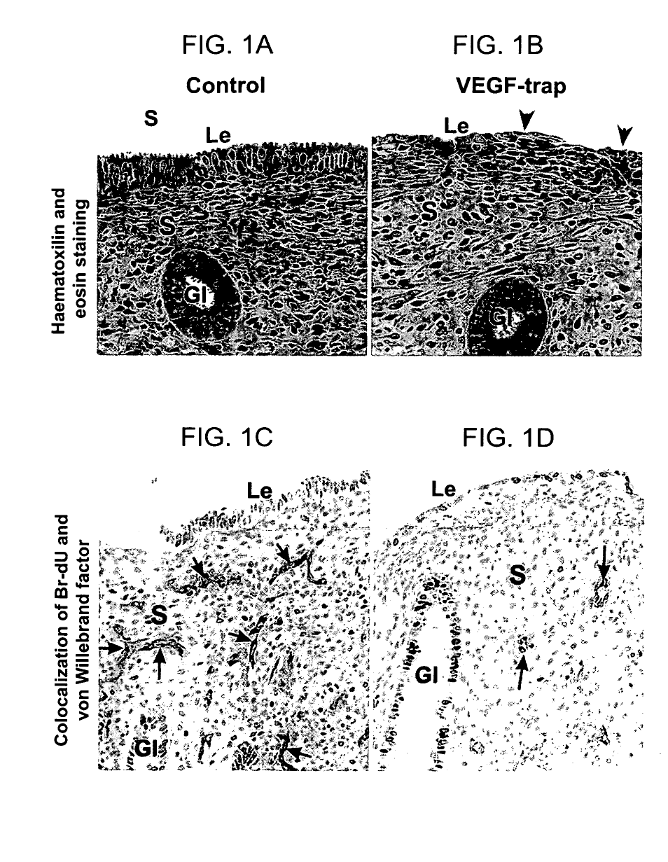

The invention is based in part on the observation that administration of a chimeric VEGF blocker (a “VEGF-trap”) polypeptide to rhesus macaques during the postmenstrual repair phase was sufficient to block subsequent vascular development and repair in the endometrium (see FIGS. 1A-D). Rhesus macaques were used for these studies because their menstrual cycle and endometrial physiology are identical to that of human females who menstruate. Since menstruation, postmenstrual healing, and regeneration of endometrium are phenomena unique to primates, such studies can not be performed in other nonprimate species.

The timing of administration of a VEGF antagonist, e.g., a VEGF-trap, is important, because administration of a VEGF antagonist, e.g., a VEGF-trap, during the vascular proliferation phase will not result in effective blockage of vascular development and endometrial repair. Most of vascular development in the primate endometrium occurs during the postmenstrual repair phase; thus, blockade of VEGF action during the later stages of the cycle (proliferative and secretory phase) will not significantly inhibit endometrial vascular development and overall endometrial development and differentiation. Thus, it was shown that administration of VEGF-trap during the post-menstrual repair phase (e.g., about 1-7 days, or about 3-5 days following the beginning of menses) is necessary and sufficient to effect blockage of vascular development and repair in the endometrium.

The effects of postmenstrual administration of a soluble VEGF blocker, VEGF-Trap, on endometrial regeneration and vascular development during the mid-proliferative phase were examined in ovariectomized, artificially cycled rhesus macaques. Six ovariectomized rhesus macaques were used. Artificial cycles were induced by sequential treatment with subcutaneous estradiol (E2) and progesterone (P) implants. At the end of one cycle, the P implant was removed and the E2 implant left in place. The animals were treated intravenously either with VEGF-Trap at a dose of 12.5 mg/kg or with vehicle on days 2, 4, and 6, and the endometrium was collected on day 8 after P withdrawal. Proliferating endothelial cells were detected through immuno-co-localization of Br-dU with von Willebrand factor (vWF), and the expression of VEGF and VEGF receptor (Flt-1 and KDR) mRNAs was examined by in situ hybridization.

VEGF-Trap treatment resulted in substantial reduction in vascular density in upper endometrial zones (FIG. 1B) leading to development of an avascular endometrium. The luminal epithelium was either atrophied or absent in most of the specimens in VEGF Trap-treated animals. A marked up-regulation of VEGF mRNA was evident in the stromal cells of the superficial zone in all the VEGF-trap treated animals. No marked differences in KDR or Flt-1 mRNA expression were evident between the endometrial vessels of control and VEGF-trap treated animals.

VEGF-Trap treatment suppressed the E2-dependent mid-proliferative peak in endometrial vascular development despite an increase in VEGF mRNA expression; The lack of vessels in the upper zones of endometrium in VEGF-Trap treated animals may have led to a localized ischemic hypoxia that upregulated VEGF mRNA in stromal cells.

Thus, VEGF-Trap blocks the postmenstrual angiogenesis and repair in the rhesus macaque endometrium.

Therapeutic Methods

The present invention provides methods of modulating endometrium, methods for inhibiting blastocyst implantation, and methods for treating a gynecological disorder resulting from abnormal endometrial growth. The methods generally involve administering an effective amount of a VEGF antagonist to an individual in need thereof. In particular embodiments, the VEGF antagonist is a chimeric VEGF blocker that functions as a VEGF antagonist. In the following description of treatment methods, a chimeric VEGF blocker is exemplified. However, any VEGF antagonist can be used in a subject method, and reference to use of a chimeric VEGF blocker in a subject method is for illustration purposes only and not meant to be limiting.

In a subject method for inhibiting pregnancy (e.g., inhibiting blastocyst implantation into the uterine wall), or for treating abnormal gynecological bleeding, an effective amount of a VEGF antagonist is administered to an individual in need thereof. In some embodiments, an “effective amount” of a VEGF antagonist (e.g., a chimeric VEGF blocker) is an amount that, when administered in one or more doses (and where administration is initiated before the mid-proliferative phase, e.g., at least a first dose is administered before the mid-proliferative phase), is effective to inhibit endometrial vascular proliferation by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the degree or extent of endometrial vascular proliferation in an untreated individual.

For example, an “effective amount” of a VEGF antagonist (e.g., a chimeric VEGF blocker) is an amount that, when administered in one or more doses (and where administration is initiated before the mid-proliferative phase, e.g., at least a first dose is administered before the mid-proliferative phase), is effective to reduce vascular development in the upper endometrial zones by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the degree or extent of vascular development in the upper endometrial zones in an untreated individual.

For example, an “effective amount” of a VEGF antagonist is an amount that, when administered in one or more doses (and where at least the first dose is administered or about day 1 of the menstrual cycle (which is established by the first day of menses), day 2 of the menstrual cycle, day 3 of the menstrual cycle, day 4 of the menstrual cycle, day 5 of the menstrual cycle, day 6 of the menstrual cycle, or day 7 of the menstrual cycle, with administration of at least a first dose on or about menstrual cycle day 1 or day 2 being of particular interest) is effective to reduce vascular development in the upper endometrial zones by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the degree or extent of vascular development in the upper endometrial zones in an untreated individual.

In some embodiments, an “effective amount” of a VEGF antagonist is an amount that, when administered in one or more doses (and where administration is initiated before the mid-proliferative phase, e.g., at least a first dose is administered before the mid-proliferative phase), is effective to reduce the thickness of the luminal epithelium by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the thickness of the luminal epithelium in an untreated individual.

VEGF Antagonists

In carrying out a subject method, an effective amount of a VEGF antagonist is administered to an individual in need thereof. Any VEGF antagonist is suitable for use in the instant invention. Suitable VEGF antagonists for use herein include agents that decrease the level of VEGF synthesis, agents that block or inhibit the binding of VEGF to a VEGF receptor (VEGFR), and agents that block or inhibit VEGF-mediated signal transduction.

Suitable VEGF antagonists include a soluble VEGF receptor that binds VEGF; a small molecule VEGF antagonist; an antibody to VEGF that blocks binding of VEGF to a VEGF receptor on a cell surface; an antibody to a VEGF receptor that blocks binding of VEGF to the receptor; and a VEGF blocker, e.g., a chimeric VEGF blocker. In some embodiments, a suitable VEGF antagonist blocks VEGF binding to a VEGF receptor on a cell. Other suitable VEGF antagonists include, but are not limited to, inhibitors of VEGFR1 tyrosine kinase activity; inhibitors of VEGFR2 tyrosine kinase activity; an antibody to VEGFR1; an antibody to VEGFR2; a ribozyme specific for VEGFR1 or VEGFR2; an antisense specific for VEGFR1 or VEGFR2; siRNA specific for VEGFR1 or VEGFR2; a soluble VEGFR; and the like.

Exemplary non-limiting VEGF antagonists that are suitable for use in a subject method include, but are not limited to, a compound as disclosed in U.S. Pat. No. 6,469,032 (e.g., 3-[(2,3-Dimethylpyrrol-5-yl)methylene]-2-indolinone, an inhibitor of VEGF-dependent phosphorylation of VEGFR-2 (known as “SU5416”; see also, Fong et al. (1999) Cancer Res. 59:99-106); 3-[2,4-dimethyl-5-(2-oxo-1,2-dihy-droindol-3-ylidenemethyl)-1H-pyrrol-3-yl]propionic acid (known as “SU6668”; an inhibitor of VEGFR-2; see, e.g., WO 99/61422; and Laird et al. (2000) Cancer Res. 60:4152); ZD4190, an inhibitor of VEGFR-1 and VEGFR-2 (see, e.g., Wedge et al. (2000) Cancer Res. 60:970; and Wedge et al. (2000) Adv. Exp. Med. Biol. 476:307-310); [N-(4-bromo-2-fluorophenyl)-6-methoxy-7-[(1-methylpiperidin-4-yl)methoxy]quinazolin-4-amine] (known as “ZD6474”; Wedge et al. (2002) Cancer Res. 62:4645); a 4-anilinoquinazoline compound as disclosed in Hennequin et al. ((2002) J. Med. Chem. 45(6):1300-12); Bevacizumab (Avastin™), a monoclonal antibody to VEGF; ZM323881 (Whittles et al. (2002) Microcirculation 9:513-522); PTK787/ZK22584, an inhibitor of VEGFR-1 and VEGFR-2 (Wood et al. (2000) Cancer Res. 60:2178-2189); Angiozyrne™, an anti-VEGFR-1 ribozyme (Weng et al. (2001) Curr. Oncol. Rep. 3:141-146); a soluble VEGFR (see, e.g., Takayama et al. (2000) Cancer Res. 60:2169-2177; Mori et al. (2000) Gene Ther. 7:1027-1033; and Mahasreshti et al. (2001) Clin. Cancer Res. 7:2057-2066); a monoclonal antibody to VEGFR-2 (see, e.g., Prewett et al. (1999) Cancer Res. 59:5209-5218; Witte et al. (1998) Cancer Metastasis Rev. 17:155-161; Brekken et al. (2000) Cancer Res. 60:5117-5124; Kunkel et al. (2001) Cancer Res. 61:6624-6628); a compound as disclosed in any of U.S. Pat. Nos. 5,792,783, 5,834,504, 5,883,113, 5,883,116, 5,886,020, 6,225,335, 6,323,228, and 6,469,032; a soluble VEGFR as disclosed in U.S. Patent Publication No. 20030181377; a compound as disclosed in U.S. Patent Publication No. 20030176487; an antibody to VEGFR as disclosed in U.S. Patent Publication No. 20030175271; a compound as disclosed in U.S. Patent Publication No. 20030171378; and the like.

In some embodiments, a suitable VEGF antagonist is an antagonist of VEGFR1 and VEGFR2. In other embodiments, a suitable VEGF antagonist is an antagonist of VEGFR1, but does not substantially antagonize VEGFR2. In other embodiments, a suitable VEGF antagonist is an antagonist of VEGF2, but does not substantially antagonist VEGFR1.

Where the VEGF antagonist is an anti-VEGFR antibody, an anti-VEGFR antibody that is suitable for use in a subject method is one that specifically binds a VEGFR (e.g., VEGFR1 and/or VEGFR2), e.g., the antibody does not substantially bind to polypeptides that lack one or more epitopes displayed by a VEGFR. Similarly, an anti-VEGF antibody that is suitable for use herein is one that specifically binds VEGF, e.g., the antibody binds VEGF but does not substantially bind to polypeptides that lack one or more epitopes displayed by a VEGF. Typically, a specific antibody is one that binds VEGF or a VEGFR with an affinity of at least about 10⁻⁷ M, at least about 10⁻⁸ M, at least about 10⁻⁹ M, or at least about 10⁻¹⁰ M, or higher.

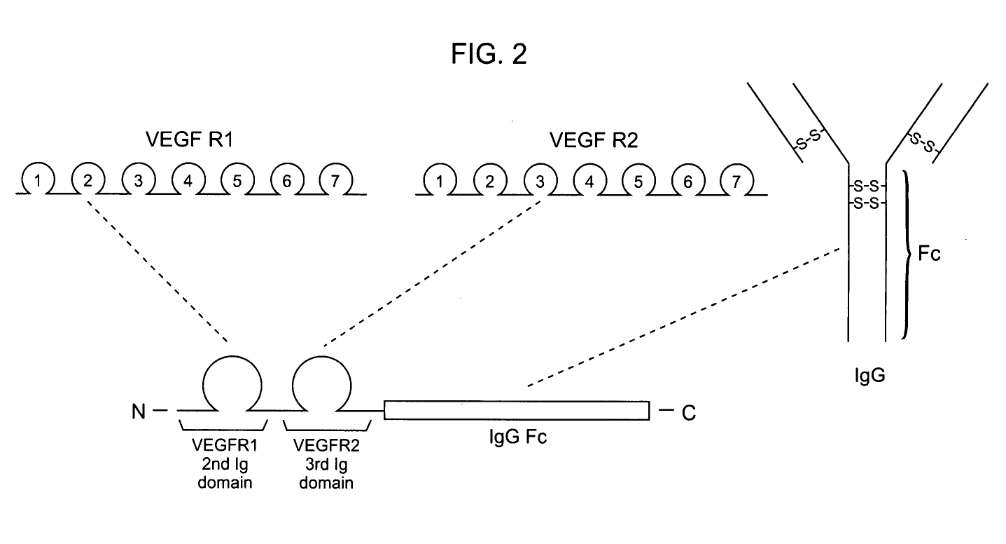

In some embodiments, the VEGF antagonist is a chimeric VEGF blocker. An exemplary chimeric VEGF blocker is depicted schematically in FIG. 2. Chimeric VEGF blocker suitable for use in a subject method includes an Ig domain from a VEGF receptor-1 (VEGFR1), an Ig domain from a VEGF receptor-2 (VEGFR2), and a dimerization domain or multimerization domain. In some embodiments, the multimerization domain is the Fc portion of an immunoglobulin (Ig). In many embodiments, a chimeric VEGF blocker suitable for use in a subject method comprises, from amino terminus to carboxyl terminus, the second Ig domain of VEGFR1, the third Ig domain of VEGFR2, and the Fc portion of human IgG1. A chimeric VEGF blocker binds VEGF, and thereby inhibits binding of VEGF to endogenous VEGF receptor. A suitable chimeric VEGF blocker lacks a VEGF receptor transmembrane domain and lacks a VEGF receptor cytoplasmic domain; therefore, a suitable chimeric VEGF receptor is soluble (e.g., is not inserted into the plasma membrane). Because it lacks a VEGF receptor transmembrane and cytoplasmic domain, binding of VEGF to a chimeric VEGF blocker does not result in signal transduction to the cytoplasm of a cell.

In some embodiments, a chimeric VEGF blocker suitable for use in a subject method includes additional amino acid sequences, including, but not limited to, polypeptides that confer a property such as ease of detection, ease of purification, etc., where examples of such polypeptides include epitope tags such as hemagglutinin, FLAG, and the like; enzymes that produce a detectable product (e.g., luciferase, β-galactosidase, horse radish peroxidase, and the like); proteins that produce a detectable signal (e.g., a green fluorescent protein); affinity tags, e.g., (His)ₙ tags such as (His)₆ for binding to Ni on affinity purification columns; immunoglobulin (Ig) domains (e.g., Fc portion of an Ig, and in particular an Fc Ig comprising human Fc Ig amino acid sequences); and binding partners for various ligands, e.g., streptavidin, etc. In other embodiments, a chimeric VEGF blocker includes a detectable label such as a fluorescent dye, a radioactive label, biotin, a hapten, etc. Fluorescent dyes include e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc.

A VEGF antagonist (e.g., a chimeric VEGF blocker) of particular interest for use in the present invention has pharmacokinetic properties that are suitable for in vivo use. In particular, a VEGF antagonist (e.g., a chimeric VEGF blocker) suitable for use in a subject method has one or more of the following properties: 1) a pI of less than about 9.4; 2) reduced binding to extracellular matrix compared to wild-type VEGFR1 or VEGFR2; 3) a maximum observed serum concentration (Cₘₐₓ) of greater than 0.05 μg/ml; and 4) an area under the serum concentration time curve (AUC) of greater than 0.04 μg×days/ml.

In some embodiments, a suitable VEGF antagonist (e.g., a chimeric VEGF blocker) exhibits one or more of the following characteristics: has an isoelectric point of less than 8.9; does not substantially bind to extracellular matrix; has a Cₘₐₓ in serum of greater than 5 μg/ml; has a Cₘₐₓ in serum of greater than 10 μg/ml; has an area under the curve in serum of greater than 10 μg×days/ml; and has an area under the curve in serum of greater than 30 μg×days/ml.

In some embodiments, a suitable VEGF antagonist (e.g., a chimeric VEGF blocker) has an isoelectric point (pI) of less than 9.4, e.g., a suitable chimeric VEGF blocker has a pI of about 9.1 or less, about 8.9 or less, or about 8.8 or less.

In some embodiments, a suitable chimeric VEGF blocker exhibits reduced binding to extracellular matrix (ECM) compared to wild-type VEGFR1 or VEGFR2, e.g., the degree of binding of a suitable chimeric VEGF blocker to ECM is reduced by about 10%, about 20%, about 30%, about 40%, about 50%, about 60%, about 70%, about 80%, about 90%, about 95%, or about 99%, compared to the binding of wild-type VEGFR1 or VEGFR2 to ECM. Whether a given chimeric VEGF blocker has reduced binding to ECM relative to the level of binding of wild-type VEGFR1 or VEGFR2 to ECM can be determined using any known ECM binding assay, including, e.g., the assay described in Holash et al. ((2002) Proc. Natl. Acad. Sci. USA 99:11393-11398). For example, ECM-coated plates are incubated for 1 hour at room temperature with various concentrations of the chimeric VEGF blocker being tested. After washing the plates to remove unbound receptor, the plates are incubated for 1 hour at room temperature with a detectably-labeled antibody specific for human Fc. After washing the plates to remove unbound antibody, the detectable label is developed, and the level of binding is determined. A reduction in the amount of bound detectable label indicates a reduction in the level of binding of the chimeric VEGF blocker to ECM.

In some embodiments, a suitable VEGF antagonist (e.g., a chimeric VEGF blocker) has a Cₘₐₓ of >0.05 μg/ml, e.g., from about 1.0 μg/ml to about 30 μg/ml or greater, e.g., a suitable soluble VEGF receptor has a Cₘₐₓ of from about 1.0 μg/ml to about 2.0 μg/ml, from about 2.0 μg/ml to about 4.0 μg/ml, from about 4.0 μg/ml to about 6.0 μg/ml, from about 6.0 μg/ml to about 8.0 μg/ml, from about 8.0 μg/ml to about 10.0 μg/ml, from about 10 μg/ml to about 12 μg/ml, from about 12 μg/ml to about 14 μg/ml, from about 14 μg/ml to about 16 μg/ml, from about 16 μg/ml to about 18 μg/ml, from about 18 μg/ml to about 20 μg/ml, from about 20 μg/ml to about 22 μg/ml, from about 22 μg/ml to about 24 μg/ml, from about 24 μg/ml to about 26 μg/ml, from about 26 μg/ml to about 28 μg/ml, or from about 28 μg/ml to about 30 μg/ml or greater.

Cₘₐₓ is determined by measuring the level of the VEGF antagonist (e.g., a chimeric VEGF blocker) in a serum sample taken from a subject at various times after administration of the VEGF antagonist to the subject. For example, where the VEGF antagonist is a chimeric VEGF blocker, the chimeric VEGF blocker is injected subcutaneously into a subject, serum samples are taken 1, 2, 4, 6, 24, 48, 72, and 144 hours after injection, and the level of chimeric VEGF blocker is measured using an enzyme-linked immunosorbent assay (ELISA). An ELISA is performed using standard methods. For example, the soluble VEGF receptor is captured onto a solid substrate coated with VEGF ligand, and the level of captured chimeric VEGF blocker is detected using a detectably labeled antibody that specifically binds to the Fc portion of human Ig. See, e.g., Holash et al. ((2002), supra).

In some embodiments, a suitable VEGF antagonist (e.g., a chimeric VEGF blocker) has an AUC of >0.04 μg×days/ml, e.g., from about 0.10 μg×days/ml to about 50 μg×days/ml or greater, e.g., a suitable VEGF antagonist (e.g., a chimeric VEGF blocker) has an AUC of from about 0.10 μg×days/ml to about 1.0 μg×days/ml, from about 1.0 μg×days/ml to about 2.5 μg×days/ml, from about 2.5 μg×days/ml to about 5.0 μg×days/ml, from about 5.0 μg×days/ml to about 7.5 μg×days/ml, from about 7.5 μg×days/ml to about 10 μg×days/ml, from about 10 μg×days/ml to about 12.5 μg×days/ml, from about 12.5 μg×days/ml to about 15 μg×days/ml, from about 15 μg×days/ml to about 17.5 μg×days/ml, from about 17.5 μg×days/ml to about 20 μg×days/ml, from about 20 μg×days/ml to about 25 μg×days/ml, from about 25 μg×days/ml to about 30 μg×days/ml, from about 30 μg×days/ml to about 35 μg×days/ml, from about 35 μg×days/ml to about 40 μg×days/ml, from about 40 μg×days/ml to about 45 μg×days/ml, from about 45 μg×days/ml to about 50 μg×days/ml, or greater. AUC is calculated from the curve generated by plotting the serum concentration of the VEGF antagonist (e.g., a chimeric VEGF blocker) over time, in days. See, e.g., Holash et al. (2002), supra).

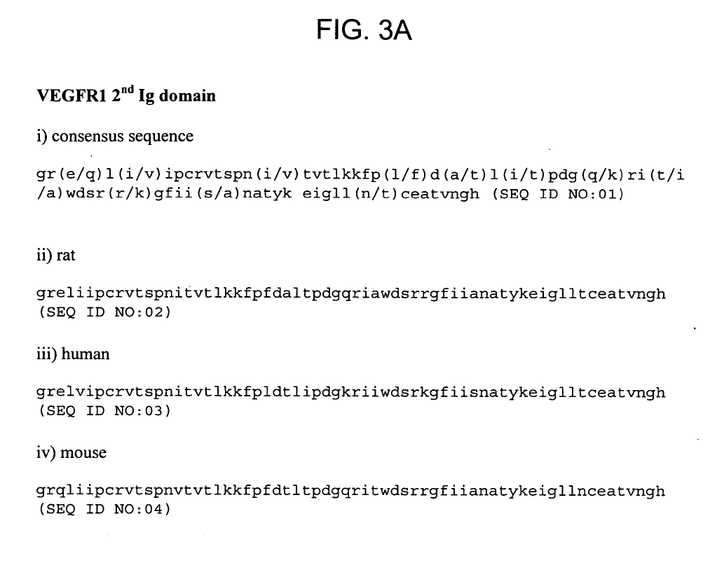

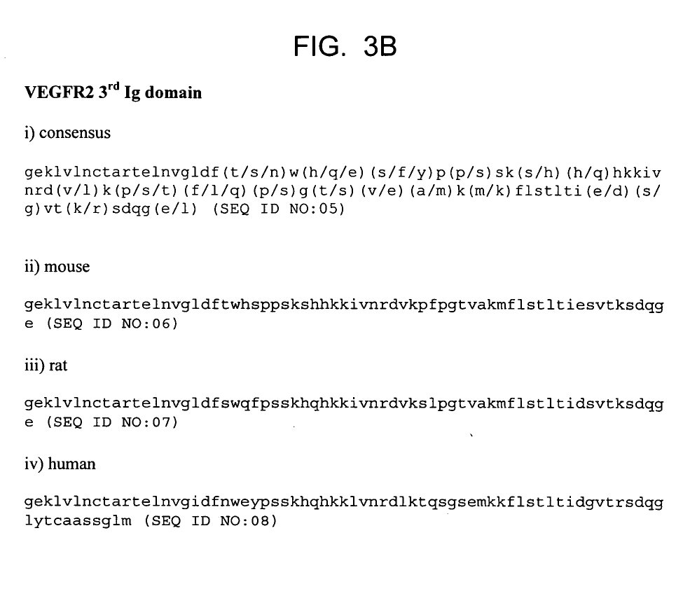

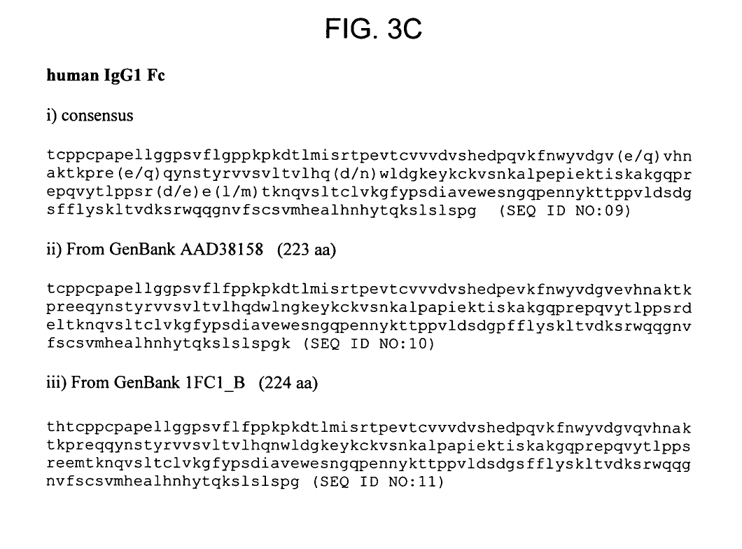

The amino acid sequences of exemplary, non-limiting portions of a suitable chimeric VEGF blocker are depicted in FIGS. 3A-C. All of the amino acid sequences disclosed herein are provided in the amino-terminus (N-terminus) to carboxyl-terminus (C-terminus) orientation.

The amino acid sequences of the second Ig domain of VEGFR1 are known in the art, and are publicly available. See, e.g., GenBank Accession No. P53767 for rat VEGFR1 amino acid sequence; GenBank Accession No. P35969 for mouse VEGFR1 amino acid sequence; and GenBank Accession No. P17948 for human VEGFR1 amino acid sequence.

Exemplary amino acid sequences of the second Ig domain of VEGFR1 are depicted in FIG. 3A. In some embodiments, the second Ig domain of VEGFR1 comprises the amino acid sequence: GR(E/Q)L(L/V)IPCRVTSPN(I/V)TVTLKKFP(L/F)D(A/T)L(I/T)PDG(Q/K)RI(T/I/A)W DSR(R/K)GFII(S/A)NATYK EIGLL(N/T)CEATVNGH (SEQ ID NO:01). This consensus sequence is based on an alignment of rat, human, and mouse VEGFR1 2^{nd} Ig domain sequences. In other embodiments, the second Ig domain of VEGFR1 comprises the amino acid sequence: GRELIIPCRVTSPNITVTLKKFPFDALTPDGQRIAWDSRRGFIIANATYKEIGLLTC EATVNGH (SEQ ID NO:02) (a rat VEGFR1 2^{nd} Ig domain sequence). In other embodiments, the second Ig domain of VEGFR1 comprises the amino acid sequence: GRELVIPCRVTSPNITVTLKKFPLDTLIPDGKRIIWDSRKGFIISNATYKEIGLLTCE ATVNGH (SEQ ID NO:03) (a human VEGFR1 2^{nd} Ig domain sequence). In other embodiments, the second Ig domain of VEGFR1 comprises the amino acid sequence: GRQLIIPCRVTSPNVTVTLKKFPFDTLTPDGQRITWDSRRGFIIANATYKEIGLLN CEATVNGH (SEQ ID NO:04) (a mouse VEGFR1 2^{nd} Ig domain sequence). In some embodiments, any of the above-mentioned VEGFR1 2^{nd} Ig domain sequences will include a further 10-25 amino acids on the N-terminus and/or the C-terminus.

The amino acid sequences of the third Ig domain of VEGFR2 are known in the art, and are publicly available. See, e.g., GenBank Accession No. P35918 for mouse VEGFR2 amino acid sequence; GenBank Accession No. 008775 for rat VEGFR2 amino acid sequence; and P35968 for human VEGFR2 amino acid sequence.

Exemplary amino acid sequences of the third Ig domain of VEGFR2 are depicted in FIG. 3B. In many embodiments, the VEGFR2 3^{rd} Ig domain comprises the following amino acid sequence: GEKLVLNCTARTELNVGLDF(T/S/N)W(H/Q/E)(S/F/Y)P(P/S)SK(S/H)(H/Q)HKKIV NRD(V/L)K(P/S/T)(F/L/Q)(P/S)G(T/S)(V/E)(A/M)K(M/K)FLSTLTI(E/D)(S/G)VT(K/R)SDQG(E/L) (SEQ ID NO:05). This consensus sequence is based on an alignment of human, mouse, and rat VEGFR2 3^{rd} Ig domain sequences. In some embodiments, the VEGFR2 3^{rd} Ig domain comprises the amino acid sequence: GEKLVLNCTARTELNVGLDFTWHSPPSKSHHKKIVNRDVKPFPGTVAKMFLSTL TIESVTKSDQGE (SEQ ID NO:06) (a mouse VEGFR2 3^{rd} Ig domain sequence). In some embodiments, the VEGFR2 3^{rd} Ig domain comprises the amino acid sequence: GEKLVLNCTARTELNVGLDFSWQFPSSKHQHKKIVNRDVKSLPGTVAKMFLST LTIDSVTKSDQGE (SEQ ID NO:07) (a rat VEGFR2 3^{rd} Ig domain sequence). In some embodiments, the VEGFR-2 3^{rd} Ig domain comprises the amino acid sequence: GEKLVLNCTARTELNVGIDFNWEYPSSKHQHKKLVNRDLKTQSGSEMKKFLST

LTIDGVTRSDQGLYTCAASSGLM (SEQ ID NO:08) (a human VEGFR2 3^{rd} Ig domain sequence). Any of the above-mentioned VEGFR2 3^{rd} Ig domain sequences can include a further 10-25 amino acids on the N-terminus and/or the C-terminus.

The amino acid sequences of the Fc portion of human Ig are known in the art and are publicly available. Of particular interest in many embodiments is the Fc portion of human IgG1. The amino acid sequence of the Fc portion of human IgG1 is found under the following GenBank accession numbers: AAD38158; 1FC1_B; 1FC1_A; P01857; and 2IG2H.

Exemplary amino acid sequences of the Fc portion of an immunoglobulin depicted in FIG. 3C. In many embodiments, an Fc portion of a chimeric VEGF blocker comprises the amino acid sequence: TCPPCPAPELLGGPSVFLGPPKPKDTLMISRTPEVTCVVVDVSHEDPQVKFNWY VDGV(E/Q)VHNAKTKPRE(E/Q)QYNSTYRVVSVLTVLHQ(D/N)WLDGKEYKCK VSNKALPEPIEKTISKAKGQPREPQVYTLPPSR(D/E)E(L/M)TKNQVSLTCLVKGF YPSDIAVEWESNGQPENNYKTTPPVLDSDGSFFLYSKLTVDKSRWQQGNVFSCS VMHEALHNHYTQKSLSLSPG (SEQ ID NO:09). This is a consensus sequence based on sequences of various human IgG Fc sequences.

In some embodiments, an Fc portion of a chimeric VEGF blocker comprises the amino acid sequence: TCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAP IEKTISKAKGQPREPQVYTLPPSRDELTKNQVSLTCLVKGFYPSDIAVEWESNGQ PENNYKTTPPVLDSDGPFFLYSKLTVDKSRWQQGNVFSCSVMHEALHNHYTQK SLSLSPG (SEQ ID NO:10). In some embodiments, the amino acid sequence of the Fc portion includes an additional Thr-His sequence on the N-terminus and/or an additional Lys residue on the C-terminus.

In some embodiments, an Fc portion of a chimeric VEGF blocker comprises the amino acid sequence: TCPPCPAPELLGGPSVFLFPPKPKDTLMISRTPEVTCVVVDVSHEDPQVKFNWYV DGVQVHNAKTKPREQQYNSTYRVVSVLTVLHQNWLDGKEYKCKVSNKALPAP IEKTISKAKGQPREPQVYTLPPSREEMTKNQVSLTCLVKGFYPSDIAVEWESNGQ PENNYKTTPPVLDSDGSFFLYSKLTVDKSRWQQGNVFSCSVMHEALHNHYTQK SLSLSPG (SEQ ID NO:11). In some embodiments, the amino acid sequence of the Fc portion includes an additional Thr-His sequence on the N-terminus and/or an additional Lys residue on the C-terminus.

The VEGFR1 2^{nd} Ig domain, VEGFR2 3^{rd} Ig domain, and Fc portion of Ig are in some embodiments fused directly to one another. In other embodiments, the VEGFR1 2^{nd} Ig domain, VEGFR2 3^{rd} Ig domain, and Fc portion of Ig are separated by one or more amino acids that serve as linkers. Proteins can be joined by a spacer peptide, generally of a flexible nature, although other chemical linkages are not excluded. Currently, it is contemplated that the most useful linker sequences will generally be peptides of between about 6 and about 40 amino acids in length, or between about 6 and about 25 amino acids in length. These linkers are generally produced by using synthetic, linker-encoding oligonucleotides to couple the proteins. Peptide linkers with a degree of flexibility will generally be preferred. The linking peptides may have virtually any amino acid sequence, bearing in mind that the preferred linkers will have a sequence that results in a generally flexible peptide. The use of small amino acids such as glycine and alanine, are of use in creating a flexible peptide. The creation of such sequences is routine to those of skill in the art. A variety of different linkers are commercially available and are considered suitable for use according to the present invention.

Amino acid sequences rich in alanine and proline residues are known to impart flexibility to multi-domain protein structures. For example, such sequences link the domains of the so-called E2 components of the 2-oxo acid dehydrogenase complexes, such as pyruvate dehydrogenase complex and 2-oxo glutarate dehydrogenase complex. Alanine-proline rich regions are also found in myosin light chains. Exemplary linkers for use in the invention have a combination of glycine, alanine, proline and methionine residues, such as AAAGGM (SEQ ID NO:12); AAAGGMPPAAAGGM (SEQ ID NO:13); AAAGGM (SEQ ID NO:14); and PPAAAGGM₂ (SEQ ID NO:15). However, any flexible linker generally between about 6 and about 40 amino acids in length may be used. Linkers may have virtually any sequence that results in a generally flexible peptide, including alanine-proline rich sequences of the type exemplified above.

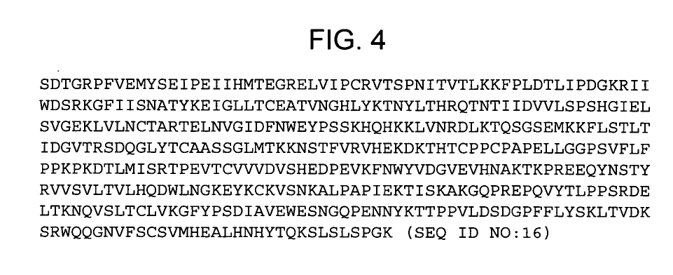

In a particular embodiment, a chimeric VEGF blocker that is suitable for use in the present invention is a chimeric VEGF blocker having an amino acid sequence as set forth in FIG. 4 (SEQ ID NO:16). This chimeric receptor is described in Holash et al. ((2002) Proc. Natl. Acad. Sci. USA 99:11393-11398) and is termed “VEGF-Trap_{R1R2}.” See also WO 02/060489. VEGF-Trap_{R1R2} includes, from amino terminus to carboxyl terminus, the second Ig domain of VEGFR1, the third Ig domain of VEGFR2, and the Fc portion of human IgG1.

Those of skill in the art will readily appreciate that changes can be made to any of the above amino acid sequences without substantially affecting the function of the polypeptide in carrying out a subject method. The amino acid sequence of the chimeric VEGF blocker polypeptide may be altered in various ways known in the art to generate targeted changes in sequence. Amino acid substitutions of interest include those that result in a reduced immune response in the individual being treated to the chimeric VEGF blocker and/or result in enhanced pharmacokinetic properties relative to wild-type VEGF receptor. A variant polypeptide will usually be substantially similar to the sequences provided herein, i.e. will differ by at least one amino acid, and may differ by at least two but not more than about ten amino acids, or not more than about twenty amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; addition of a fatty acid or lipid; changes in amino acid sequence that make the protein susceptible to PEGylation (e.g., covalent linkage of one or more polyethylene glycol moieties); and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability, e.g., a polyethylene glycol (PEG) moiety may be attached, directly or via a linker, to one or more amino acids in the soluble chimeric VEGF blocker polypeptide.

Polyethylene glycol suitable for linkage to a chimeric VEGF blocker polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG moiety may be linear or branched, and generally has a molecular weight in a range of from about 2 kDa to about 100 kDa. The PEG moiety may have at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine. The PEG moiety may also be derivatized so that it is reactive with free carboxyl groups or with free amino groups.

PEG may be attached to the chimeric VEGF blocker via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group, an epoxide group, an oxycarbonylimidazole group, a nitro phenyl group, a trysylate group, an aldehyde group, an isocyanate group, a vinylsulfone group, a tyrosine group, a cysteine group, a histidine group or a primary amine.

The chimeric VEGF blocker polypeptides may be prepared by in vitro synthesis, using conventional methods as known in the art, by recombinant methods, or may be isolated from cells induced to produce the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like. In some embodiments, a VEGF blocker polypeptide is produced by recombinant methods, using eukaryotic cells. In some embodiments, a VEGF blocker polypeptide is produced by recombinant methods, using prokaryotic cells.

The VEGF antagonist polypeptides may also be isolated and purified in accordance with conventional methods of recombinant synthesis. A lysate may be prepared of the expression host and the lysate purified using high performance liquid chromatography, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. In many embodiments, a VEGF antagonist suitable for use in a subject method is pure, e.g., at least about 90%, at least about 95%, at least about 98%, or at least about 99%, or higher, pure.

Methods for Inhibiting Pregnancy

The present invention provides methods of preventing or inhibiting pregnancy in a female human of child-bearing age, the method generally involving administering to the female human an effective amount of a VEGF antagonist, e.g., a chimeric VEGF blocker, in a time period such that implantation of the blastocyst is inhibited. Suitable time periods, doses, dosages, and routes of administration are discussed in detail below.

In carrying out a subject contraceptive method, an effective amount of a VEGF antagonist, e.g., a chimeric VEGF blocker, is administered. An effective amount of a VEGF antagonist (e.g., a chimeric VEGF blocker) is an amount that inhibits blastocyst implantation, e.g, an amount that is at least about 90%, at least about 95%, at least about 97%, at least about 98%, at least about 99% effective, or 100% effective, in inhibiting blastocyst implantation. That is, in at least about 90%, at least about 95%, at least about 97%, at least about 98%, or at least about 99%, or 100% of the times that fertilization of an egg occurs, implantation does not occur, and no further development of the blastocyst ensues.

Methods for Treating Gynecological Bleeding Disorders

The present invention provides methods of treating a gynecological bleeding disorder in a female human in need thereof, the method generally involving administering to the individual an effective amount of a VEGF antagonist (e.g., a chimeric VEGF blocker) in a time period suitable for effect a reduction in gynecological bleeding. Suitable time periods, dosages, and routes of administration are discussed in detail below.

Forms of dysfunctional uterine bleeding (dysfunctional or abnormal uterine bleeding, metrorrhagias and menorrhagias, hypermenorrhea) that are treated using a subject method are forms of pathological bleeding that are not attributable to organic changes in the uterus (such as, e.g., endometrial carcinoma, myomas, polyps, etc.), systemic coagulation disorders, or a pathological pregnancy (e.g., ectopic pregnancy, impending spontaneous abortion). The average blood loss during normal menstruation is about 30 ml, whereby the period lasts for an average of 5 days. If the blood loss exceeds about 80 ml, it is classified as pathological.

Metrorrhagias are defined as bleeding that may or may not be accompanied by pain and that cannot be linked to menstruation or cycle. If it lasts over 7 days, the blood loss often exceeds 80 ml. Menorrhagia is menstruation that may or may not be accompanied by pain, normally every 27-28 days, which, when it lasts over 7 days, is associated in most cases with an increased blood loss of over 80 ml. Hypermenorrhea is defined as menstruation that may or may not be accompanied by pain, normally about every 27-28 days for about 4-5 days with an elevated blood loss of over 80 ml.

In carrying out a subject method of treating a dysfunctional gynecological bleeding disorder, an effective amount of a VEGF antagonist (e.g., a chimeric VEGF blocker) is administered. An effective amount of a VEGF antagonist is an amount that reduces excessive gynecological blood loss by at least about 10%, at least about 20%, at least about 30%, at least about 40%, at least about 50%, at least about 60%, at least about 70%, at least about 80%, or at least about 90%, or more, when compared to the amount of gynecological blood loss in excess of 80 ml that occurs in the absence of treatment with the VEGF antagonist.

Formulations, Dosages, and Routes of Administration

An active agent (e.g., a VEGF antagonist, e.g., a chimeric VEGF blocker polypeptide) is administered to individuals in a formulation with a pharmaceutically acceptable excipient(s). The terms “agent,” “active agent,” “therapeutic agent,” and “drug” are used interchangeably herein. A wide variety of pharmaceutically acceptable excipients are known in the art and need not be discussed in detail herein.

Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; Remington's Pharmaceutical Sciences, 19th Edition, A. R. Gennaro, ed., (1995 or latest edition); and Handbook of Pharmaceutical Excipients (2000) A. H. Kibbe et al., eds., 3^{rd} ed. Amer. Pharmaceutical Assoc.

Agents can be administered locally, topically, enterally, transdermally, intravaginally, subcutaneously, intravenously, intramuscularly, orally, or parenterally. For oral administration, especially tablets, coated tablets, capsules, pills, suspensions, or solutions are suitable, which can be produced in the usual way with the additives and vehicles that are commonly used in galenicals. For local or topical application, for example, vaginal suppositories, vaginal gels, implants, vaginal sponges, vaginal rings, or tampons; are suitable. For transdermal application, transdermal systems such as skin patches are suitable.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacterial and fungi. The carrier can be a solvent or dispersion medium containing, for example, water, ethanol (e.g., glycerol, propylene glycol and liquid polyethylene glycol), suitable mixtures thereof, and vegetable oil.

An active agent (e.g., a VEGF antagonist; e.g., a chimeric VEGF blocker polypeptide) may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

A subject formulation can be prepared by the methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Dosages

Effective amounts of an active agent (e.g., a VEGF antagonist, e.g., a chimeric VEGF blocker polypeptide) will vary, depending on a variety of factors, including, e.g., the desired therapeutic effect (e.g., contraception, or reduction in abnormal gynecological bleeding); the pharmacokinetics of the particular agent (e.g., serum half-life); the extent, degree, or severity of the condition being treated (e.g., the extent of abnormal gynecological bleeding); the age of the individual being treated; the overall health status of the individual being treated; etc. Such factors are well within the skill level of medical personnel, or other person skilled in the art, to determine.

Effective amounts of a VEGF antagonist generally range from about 10 mg to about 1500 mg per dose, e.g., from about 10 mg per dose to about 25 mg per dose, from about 25 mg per dose to about to about 50 mg per dose, from about 50 mg to about 75 mg per dose, from about 75 mg to about 100 mg per dose, from about 100 mg per dose to about 150 mg per dose, from about 150 mg per dose to about 200 mg per dose, from about 200 mg per dose to about 250 mg per dose, from about 250 mg per dose to about 300 mg per dose, from about 300 mg per dose to about 350 mg per dose, from about 350 mg per dose to about 400 mg per dose, from about 400 mg per dose to about 450 mg per dose, from about 450 mg per dose to about 500 mg per dose, from about 500 mg per dose to about 600 mg per dose, from about 600 mg per dose to about 700 mg per dose, from about 700 mg per dose to about 800 mg per dose, from about 800 mg per dose to about 900 mg per dose, from about 900 mg per dose to about 1000 mg per dose, from about 1000 mg per dose to about 1100 mg per dose, from about 1100 mg per dose to about 1200 mg per dose, from about 1200 mg per dose to about 1300 mg per dose, from about 1300 mg per dose to about 1400 mg per dose, or from about 1400 mg per dose to about 1500 mg per dose.

In some embodiments, effective dosages of a VEGF antagonist are expressed as mg per body surface area (mg/m²). In these embodiments, effective dosages of a VEGF antagonist are from about 10 mg/m² to about 500 mg/m², e.g., from about 10 mg/m² to about 25 mg/m², from about 25 mg/m² to about 50 mg/m², from about 50 mg/m² to about 75 mg/m², from about 75 mg/m² to about 100 mg/m², from about 100 mg/m² to about 125 mg/m², from about 125 mg/m² to about 150 mg/m², from about 150 mg/m² to about 175 mg/m², from about 175 mg/m² to about 200 mg/m², from about 225 mg/m², from about 225 mg/m² to about 250 mg/m², from about 250 mg/m² to about 300 mg/m², from about 300 mg/m² to about 350 mg/m², from about 350 mg/m² to about 400 mg/m², from about 400 mg/m² to about 450 mg/m², or from about 450 mg/m² to about 500 mg/m².

In some embodiments, effective dosages of a VEGF antagonist are expressed as mg/kg body weight. In these embodiments, effective dosages of a VEGF antagonist are from about 5 mg/kg body weight to about 200 mg/kg body weight, e.g., from about 5.0 mg/kg body weight to about 10 mg/kg body weight, from about 10 mg/kg body weight to about 15 mg/kg body weight, from about 15 mg/kg body weight to about 25 mg/kg body weight, from about 25 mg/kg body weight to about 50 mg/kg body weight, from about 50 mg/kg body weight to about 75 mg/kg body weight, from about 75 mg/kg body weight to about 100 mg/kg body weight, from about 100 mg/kg body weight to about 125 mg/kg body weight, from about 125 mg/kg body weight to about 150 mg/kg body weight, or from about 150 mg/kg body weight to about 200 mg/kg body weight.

In general, a unit dosage amount of a chimeric VEGF blocker polypeptide will range from about 0.05 mg/kg to about 20 mg/kg, e.g., from about 0.05 mg/kg to about 0.1 mg/kg, from about 0.1 mg/kg to about 0.5 mg/kg, from about 0.5 mg/kg to about 0.75 mg/kg, from about 0.75 mg/kg to about 1.0 mg/kg, from about 1.0 mg/kg to about 1.2 mg/kg, from about 1.2 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, from about 4.0 mg/kg to about 4.5 mg/kg, from about 4.5 mg/kg to about 5.0 mg/kg, from about 5.0 mg/kg to about 5.5 mg/kg, from about 5.5 mg/kg to about 6.0 mg/kg, from about 6.0 mg/kg to about 6.5 mg/kg, from about 6.5 mg/kg to about 7 mg/kg, from about 7 mg/kg to about 8 mg/kg, from about 8 mg/kg to about 9 mg/kg, from about 9 mg/kg to about 10 mg/kg, from about 10 mg/kg to about 12.5 mg/kg, from about 12.5 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, or from about 40 mg/kg to about 50 mg/kg.

As used herein, the terms “day 2 of the menstrual cycle” and “two days following the beginning of the menstrual cycle,” are used interchangeably. Day 1 of the menstrual cycle is the first day of menses. A typical menstrual cycle is 28 days, although a normal menstrual cycle can vary from 21 days to 35 days. Thus, the term, “menstrual cycle,” as used herein, includes normal variations from the 28 day cycle, e.g., 27 days, 30 days, etc. Those skilled in the art will recognize that the descriptions of dosages described herein can be modified to accommodate normal variations in menstrual cycles.

A suitable time period for administering at least a first dose of the VEGF antagonist includes, e.g., a time period of from about day 1 to about day 7 of the menstrual cycle (where day 1 is defined as the first day of menses); a time period of from about day 3 to about day 5 of the menstrual cycle; a time period of from about day 1 to about day 7 of the menstrual cycle; a time period of from about day 2 to about day 7 of the menstrual cycle (where day 1 is defined as the first day of menses); a time period of from about day 2 to about day 5 of the menstrual cycle; a time period of from about day 2 to about day 7 of the menstrual cycle; etc. In any event, administration of a VEGF antagonist should begin before the mid-proliferative phase, e.g., at least a first dose of a VEGF antagonist is administered before the mid-proliferative phase, e.g, before day 10 of the menstrual cycle.

In some embodiments, a single dose of a VEGF antagonist (e.g., a chimeric VEGF blocker) is delivered (administered). The following are non-limiting examples. In some embodiments, a single dose of a VEGF antagonist (e.g., a chimeric VEGF blocker) is delivered at the start of menses. In some embodiments, a single dose of a VEGF antagonist (e.g., a chimeric VEGF blocker) is delivered on about day 1 of the menstrual cycle (the day of the start of menses). In some embodiments, a single dose of a VEGF antagonist (e.g., a chimeric VEGF blocker) is delivered on about day 2 of the menstrual cycle. In some embodiments, a single dose of a VEGF antagonist (e.g., a chimeric VEGF blocker) is delivered on about day 3 of the menstrual cycle. In some embodiments, a single dose of a VEGF antagonist (e.g., a chimeric VEGF blocker) is delivered on about day 4 of the menstrual cycle. In some embodiments, a single dose of a VEGF antagonist (e.g., a chimeric VEGF blocker) is delivered on about day 5 of the menstrual cycle. In some embodiments, a single dose of a VEGF antagonist (e.g., a chimeric VEGF blocker) is delivered on about day 6 of the menstrual cycle. In some embodiments, a single dose of a VEGF antagonist (e.g., a chimeric VEGF blocker) is delivered on about day 7 of the menstrual cycle.

In some embodiments, multiple doses of a VEGF antagonist (e.g., a chimeric VEGF blocker) are delivered Whether a single dose of a VEGF antagonist will be effective to achieve a therapeutic goal (e.g., inhibition of pregnancy; reduction in abnormal gynecological bleeding; etc.) will depend on various factors, including, e.g., the pharmacokinetic profile of the particular VEGF antagonist (e.g., the serum half-life of the VEGF antagonist); the bioavailability of the VEGF antagonist; the IC₅₀ of the VEGF antagonist; and the like.

Where multiple doses are administered over a period of time a VEGF antagonist is administered twice daily (qid), daily (qd), every other day (qod), every third day, three times per week (tiw), or twice per week (biw) over a period of time. For example, a VEGF antagonist (e.g., a chimeric VEGF blocker) is administered qid, qd, qod, tiw, or biw over a period of from one day to about 2 weeks, e.g., from about one day to about 2 days, from about 2 days to about 3 days, from about 3 days to about 4 days, from about 4 days to about 5 days, from about 5 days to about 7 days, or from about 7 days to about 14 days.

In some embodiments, a VEGF antagonist (e.g., a chimeric VEGF blocker) is administered qid, qd, qod,.tiw, or biw over a period of from one day to about 2 weeks, e.g., from about one day to about 2 days, from about 2 days to about 3 days, from about 3 days to about 4 days, from about 4 days to about 5 days, from about 5 days to about 7 days, or from about 7 days to about 14 days, where the VEGF antagonist therapy regimen begins on the day menses starts, or beginning on a day. chosen from day 1 to about day 5 of the menstrual cycle.

In many embodiments, the individual to whom the VEGF antagonist is administered is an individual has not received the VEGF antagonist for a period of time of from about 5 days to about 27 days, e.g., the VEGF antagonist has not been administered to the individual for a period of time of from about 5 days to about 7 days, from about 7 days to about 10 days, from about 10 days to about 14 days, from about 14 days to about 21 days, or from about 21 days to about 27 days, before the VEGF antagonist treatment. For example, where a VEGF antagonist is administered once during a menstrual cycle, and the VEGF antagonist is administered on day 2 of the menstrual cycle, the individual has not received the VEGF antagonist for a period of time of about 27 days prior to administration of the VEGF antagonist at day 2 of the menstrual cycle.

In some embodiments, multiple doses of a VEGF antagonist (e.g., a chimeric VEGF blocker) are delivered. In some embodiments, a first dose of a VEGF antagonist (e.g., a chimeric VEGF blocker) is delivered at the beginning of menses; and at least a second dose of the VEGF antagonist is delivered on a second day chosen from any day within from about day one to about day 14 of the menstrual cycle. At least one further dose will be administered in some embodiments, where the at least one further dose is administered on a day chosen from about one to about 14 days following the previous dose, e.g., to provide for administration of a second dose on about day 14 of the menstrual cycle. In other embodiments, a first dose of a VEGF antagonist (e.g., a chimeric VEGF blocker) is delivered on a day chosen from about day one to about day 7 of the menstrual cycle; and a second dose of the VEGF antagonist is delivered on a second day that is from one day to about 14 days following the first dose, e.g., on day 14 of the menstrual cycle. In other embodiments, a first dose of a VEGF antagonist (e.g., a chimeric VEGF blocker) is delivered on a day chosen from about day one to about day 7 of the menstrual cycle; a second dose of the VEGF antagonist is delivered on a second day that is from one day to about 14 days following the first dose (e.g., on about day 14 of the menstrual cycle); and a third dose of the VEGF antagonist is delivered on a second day that is from one day to about 14 days following the second dose. In some embodiments, one or more further dose, e.g., a fourth dose, a fifth dose, a sixth dose, etc., is(are) administered on a day that is from one day to about 7 days following the previous dose.

In some embodiments, a first dose of a VEGF antagonist (e.g., a chimeric VEGF blocker) is delivered at the beginning of menses; and a second dose of the VEGF antagonist is delivered on a second day chosen from any day within from about day one to about day 14 of the menstrual cycle. The following are non-limiting examples. In some embodiments, a first dose of a VEGF antagonist (e.g., a chimeric VEGF blocker) is delivered at or near the beginning of menses (e.g., on day 1 or day 2 of the menstrual cycle); and a second dose of the VEGF antagonist is delivered on day two of the menstrual cycle. In some embodiments, a first dose of a VEGF antagonist (e.g., a chimeric VEGF blocker) is delivered on day one of the menstrual cycle; and a second dose of the VEGF antagonist is delivered on day three of the menstrual cycle. In some embodiments, a first dose of a VEGF antagonist (e.g., a chimeric VEGF blocker) is delivered at or near the beginning of menses (e.g., on day 1 or day 2); and a second dose of the VEGF antagonist is delivered on day four of the menstrual cycle. In some embodiments, a first dose of a VEGF antagonist (e.g., a chimeric VEGF blocker) is delivered at or near the beginning of menses (e.g., on day 1 or day 2); and a second dose of the VEGF antagonist is delivered on day five of the menstrual cycle. In some embodiments, a first dose of a VEGF antagonist (e.g., a chimeric VEGF blocker) is delivered at or near the beginning of menses (e.g., on day 1 or day 2); and a second dose of the VEGF antagonist is delivered on day six of the menstrual cycle. In some embodiments, a first dose of a VEGF antagonist (e.g., a chimeric VEGF blocker) is delivered at or near the beginning of menses (e.g., on day 1 or day 2); and a second dose of the VEGF antagonist is delivered on day seven of the menstrual cycle. In some embodiments, a first dose of a VEGF antagonist (e.g., a chimeric VEGF blocker) is delivered at or near the beginning of menses (e.g., on day 1 or day 2); and a second dose of the VEGF antagonist is delivered on day 14 of the menstrual cycle.

In some embodiments, a first dose of a VEGF antagonist (e.g., a chimeric VEGF blocker) is delivered on a day chosen from about day one to about day 7 of the menstrual cycle; and a second dose of the VEGF antagonist is delivered on a second day that is from one day to about 14 days following the first dose. The following are non-limiting examples. In some embodiments, a first dose of a VEGF antagonist (e.g., a chimeric VEGF blocker) is delivered on day 2 of the menstrual cycle; and a second dose of the VEGF antagonist is delivered on day 14 of the menstrual cycle. In some embodiments, a first dose of a VEGF antagonist (e.g., a chimeric VEGF blocker) is delivered on day one of the menstrual cycle; and a second dose of the VEGF antagonist is delivered on day 14 of the menstrual cycle. In some embodiments, a first dose of a VEGF antagonist (e.g., a chimeric VEGF blocker) is delivered on day two of the menstrual cycle; and a second dose of the VEGF antagonist is delivery on day four of the menstrual cycle. In some embodiments, a first dose of a VEGF antagonist (e.g., a chimeric VEGF blocker) is delivered on day 2 of the menstrual cycle; and a second dose of the VEGF antagonist is delivered on day 7 of the menstrual cycle. In some embodiments, a first dose of a VEGF antagonist (e.g., a chimeric VEGF blocker) is delivered on day one of the menstrual cycle; and a second dose of the VEGF antagonist is delivered on day 7 of the menstrual cycle.

In other embodiments, a first dose of a VEGF antagonist (e.g., a chimeric VEGF blocker) is delivered on a day chosen from about day one to about day 7 of the menstrual cycle; a second dose of the VEGF antagonist is delivered on a second day that is from one day to about 14 days following the first dose; and a third dose of the VEGF antagonist is delivered on a second day that is from one day to about 14 days following the second dose. The following are non-limiting examples. In some embodiments, a first dose of a VEGF antagonist (e.g., a chimeric VEGF blocker) is delivered on day 2 of the menstrual cycle; a second dose of the VEGF antagonist is delivered on day 7 of the menstrual cycle; and a third dose of the VEGF antagonist is delivered on day 14 of the menstrual cycle. In some embodiments, a first dose of a VEGF antagonist (e.g., a chimeric VEGF blocker) is delivered on day one of the menstrual cycle; a second dose of the VEGF antagonist is delivered on day 7 of the menstrual cycle; and a third dose of the VEGF antagonist is delivered on day 14 of the menstrual cycle.

In some embodiments, a VEGF antagonist (e.g., a chimeric VEGF blocker) is administered continuously or substantially continuously. In some embodiments, a VEGF antagonist (e.g., a chimeric VEGF blocker) is administered continuously or substantially continuously from the beginning of menses on day 1 through a day chosen from day 2 to about day 14 of the menstrual cycle. In other embodiments, a VEGF antagonist (e.g., a chimeric VEGF blocker) is administered continuously or substantially continuously from a day chosen from day 1 to about day 5 through a day chosen from day 1 to about day 14 following initiation of VEGF antagonist treatment.

In some embodiments, a VEGF antagonist is administered continuously or substantially continuously from the beginning of menses through a day chosen from day 1 to about day 14 of the menstrual cycle. The following are non-limiting examples. In some embodiments, a VEGF antagonist is administered continuously or substantially continuously from the beginning of menses through day 1 of the menstrual cycle. The following are non-limiting examples. In some embodiments, a VEGF antagonist is administered continuously or substantially continuously from the beginning of menses through day 2 of the menstrual cycle. The following are non-limiting examples. In some embodiments, a VEGF antagonist is administered continuously or substantially continuously from the beginning of menses through day 4 of the menstrual cycle. The following are non-limiting examples. In some embodiments, a VEGF antagonist is administered continuously or substantially continuously from the beginning of menses through day 6 of the menstrual cycle. The following are non-limiting examples. In some embodiments, a VEGF antagonist is administered continuously or substantially continuously from the beginning of menses through day 7 of the menstrual cycle. The following are non-limiting examples. In some embodiments, a VEGF antagonist is administered continuously or substantially continuously from the beginning of menses through day 14 of the menstrual cycle.

In some embodiments, a VEGF antagonist (e.g., a chimeric VEGF blocker) is administered continuously or substantially continuously from a day chosen from day 1 to about day 5 of the menstrual cycle, through a day chosen from day 1 to about day 14 following initiation of VEGF antagonist treatment. The following are non-limiting examples. In some embodiments, a VEGF antagonist (e.g., a chimeric VEGF blocker) is administered continuously or substantially continuously from day 1 of the menstrual cycle through day 5 of the menstrual cycle. In some embodiments, a VEGF antagonist (e.g., a chimeric VEGF blocker) is administered continuously or substantially continuously from day 1 of the menstrual cycle through day 7 of the menstrual cycle. In some embodiments, a VEGF antagonist (e.g., a chimeric VEGF blocker) is administered continuously or substantially continuously from day 1 of the menstrual cycle through day 14 of the menstrual cycle. In some embodiments, a VEGF antagonist (e.g., a chimeric VEGF blocker) is administered continuously or substantially continuously from day 2 of the menstrual cycle through day 5 of the menstrual cycle. In some embodiments, a VEGF antagonist (e.g., a chimeric VEGF blocker) is administered continuously or substantially continuously from day 2 of the menstrual cycle through day 7 of the menstrual cycle. In some embodiments, a VEGF antagonist (e.g., a chimeric VEGF blocker) is administered continuously or substantially continuously from day 2 of the menstrual cycle through day 14 of the menstrual cycle.

Routes of Administration

In some embodiments, a VEGF antagonist (e.g., a chimeric VEGF blocker) is delivered to the uterus by intravaginal administration of the VEGF antagonist. In other embodiments, a VEGF antagonist (e.g., a chimeric VEGF blocker) is delivered by systemic administration of chimeric VEGF blocker. In some embodiments, a VEGF antagonist is delivered orally. In some embodiments, a VEGF antagonist (e.g., a chimeric VEGF blocker) is delivered by bolus intravenous injection. In some embodiments, a VEGF antagonist (e.g., a chimeric VEGF blocker) is delivered by bolus subcutaneous injection. In some embodiments, a VEGF antagonist (e.g., a chimeric VEGF blocker) is delivered by intramuscular injection. In other embodiments, a VEGF antagonist (e.g., a chimeric VEGF blocker) is delivered transdermally, e.g., using a patch or other transdermal delivery device or system.

Transvaginal Delivery of a VEGF Antagonist

In some embodiments, a subject method provides for the transvaginal delivery of a VEGF antagonist (e.g., a chimeric VEGF blocker) to the uterus, myometrium, or endometrium of a female human. In these embodiments, the methods generally involve delivering a pharmaceutical formulation comprising a VEGF antagonist (e.g., a chimeric VEGF blocker) into the vagina of the individual. The VEGF antagonist is introduced into the vagina using an intravaginal device inserted into vagina. For example, an intravaginal nng, sponge, vaginal applicator, vaginal suppository, tampon or tampon-like device that comprises a formulation comprising a VEGF antagonist (e.g., a chimeric VEGF blocker) formulated as a bioadhesive microparticle, an intravaginal cream, an intravaginal lotion, an intravaginal foam, an intravaginal paste, an intravaginal ointment, an intravaginal solution or an intravaginal gel is inserted into the vagina.

In some embodiments, in addition to a VEGF antagonist (e.g., a chimeric VEGF blocker), the formulation will include one or more of a pharmaceutically acceptable, non-toxic excipient which promotes delivery of the pharmaceutical agent through the vaginal epithelium; a mucoadhesive agent; and a penetration enhancing agent. Mucoadhesive agents suitable for use include, but are not limited to, an alginate; pectin; a cellulose derivative, e.g., hydroxymethyl propylcellulose; and the like. Non-toxic excipients include, but are not limited to, a mixture of semisynthetic glycerides of saturated fatty acids of eight to eighteen carbons. Penetration enhancing agents suitable for use include, but are not limited to, a bile salt, an organic solvent, ethoxydiglycol, and an esterified oil.

In some embodiments, the formulation further includes a nonsteroidal anti-inflammatory drug. Suitable nonsteroidal anti-inflammatory drugs include, but are not limited to, Aspirin, Ibuprofen, Indomethacin, Phenylbutazone, Bromfenac, Fenamate, Sulindac, Nabumetone, Ketorolac, and Naproxen.

Systemic Administration of a VEGF Antagonist

In some embodiments, a VEGF antagonist (e.g., a chimeric VEGF blocker) is delivered systemically to the individual. In some embodiments, a VEGF antagonist (e.g., a chimeric VEGF blocker) is injected subcutaneously. In other embodiments, a VEGF antagonist (e.g., a chimeric VEGF blocker) is injected intravenously. In other embodiments, a VEGF antagonist (e.g., a chimeric VEGF blocker) is delivered transdermally. In other embodiments, a VEGF antagonist (e.g., a chimeric VEGF blocker) is injected intramuscularly. In these embodiments, the VEGF antagonist is formulated for injection, e.g., the VEGF antagonist is formulated in a liquid solution that includes at least one pharmaceutically acceptable excipient suitable for administration by injection.

Combination Therapies

In some embodiments, a subject method for inhibiting pregnancy comprises administration of a VEGF antagonist, e.g., a chimeric VEGF blocker; and a second agent that is suitable for inhibiting pregnancy. Where a subject method is a method for inhibiting pregnancy by inhibiting implantation of a blastocyst in the uterine walk, the combination treatment comprises administration of a VEGF antagonist (e.g., a chimeric VEGF blocker) and a second agent that inhibits contraception, implantation, or further development of a fertilized egg. In some embodiments, a subject method for treating abnormal gynecological bleeding comprises administering a VEGF antagonist (e.g., a chimeric VEGF blocker) and a second therapeutic agent for the treatment of abnormal gynecological bleeding.

The VEGF antagonist and the second agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In some embodiments, the additional agent(s) is administered during the entire course of VEGF antagonist (e.g., chimeric VEGF blocker) treatment, and the beginning and end of the treatment periods coincide. In other embodiments, the additional agent(s) is administered for a period of time that is overlapping with that of the a VEGF antagonist (e.g., a chimeric VEGF blocker) treatment, e.g., treatment with the additional therapeutic agent(s) begins before the chimeric VEGF blocker treatment begins and ends before the VEGF antagonist treatment ends; treatment with the additional therapeutic agent(s) begins after the VEGF antagonist treatment begins and ends after the VEGF antagonist treatment ends; treatment with the additional therapeutic agent(s) begins after the VEGF antagonist treatment begins and ends before the VEGF antagonist treatment ends; or treatment with the additional therapeutic agent(s) begins before the VEGF antagonist treatment begins and ends after the VEGF antagonist treatment ends.

Combination Therapy for Inhibiting Pregnancy

In some embodiments, a VEGF antagonist (e.g., a chimeric VEGF blocker) is administered in conjunction with second agent for inhibiting pregnancy. Suitable second agents include, but are not limited to, an agent such as a progestin, an antiprogestogen, and/or an estrogen. Progestiris are known in the art and include, but are not limited to, Norgestimate, Norethindrone, Norgestrel, Desogestrel, Norethindrone acetate, and Levonorgestrel. Suitable second agents also include antagonists of the progesterone receptor such as those- described in U.S. Pat. No. 6,503,939. Suitable second agents also include progesterone antagonists such as those described in U.S. Pat. No. 6,451,780.

In some embodiments, a subject combination treatment for inhibiting pregnancy comprises administration of a chimeric VEGF blocker and administration of a progestin. In other embodiments, a subject combination treatment for inhibiting pregnancy comprises administration of a chimeric VEGF blocker; administration of a progestin; and administration of an estrogen.

Norgestimate (NGM), ethinyl estradiol (EE) and 17-β estradiol (E₂) are contraceptive progestin and estrogen steroids known as active agents in oral contraceptive tablet combination formulations. NGM in combination with EE is marketed under the trademark Tricyclen® in a triphasic package containing tablets having 180, 215 and 250 μg NGM dosage strengths in combination with 35 μg EE. In addition, a monophasic package containing tablets having 250 μg NGM in combination with 35 μg EE is marketed under the trademark Cyclen®. In addition, a hormone replacement therapy formulation approved for marketing under the trademark Prefest® contains tablets having 90 μg NGM in combination with 1 mg E₂.

Any ofthe above-described treatment regimens described comprising administering a VEGF antagonist (e.g., a chimeric VEGF blocker) can be modified to include administration of a progestin. In some embodiments, the methods further comprise administration of an estrogen. The following are non-limiting examples of treatment regimens.

In a particular embodiment, a subject combination treatment for inhibiting pregnancy comprises subcutaneous administration by injection of from about 0.05 mg/kg to about 20 mg/kg VEGF antagonist on about day 1 or on about day 2 of the menstrual cycle; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg VEGF antagonist on about day 1 or on about day 2 of the menstrual cycle; and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg VEGF antagonist on about day 1 or on about day 2 of the menstrual cycle; and administration of a progestin. In some of these embodiments, the methods further comprise administration of an estrogen.

In a particular embodiment, a subject combination treatment for inhibiting pregnancy comprises subcutaneous administration by injection of from about 0.05 mg/kg to about 20 mg/kg VEGF antagonist on about day 2 and about day 14 of the menstrual cycle; and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg VEGF antagonist on day 2 and day 14 of the menstrual cycle; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg VEGF antagonist on about day 2 and about day 14 of the menstrual cycle; and administration of a progestin. In some of these embodiments, the methods further comprise administration of an estrogen.

In a particular embodiment, a subject combination treatment for inhibiting pregnancy comprises subcutaneous administration by injection of from about 0.05 mg/kg to about 20 mg/kg VEGF antagonist over a period of time of from about 2 to about 7 days beginning on the day of menses to 5 days following the start of menses; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg VEGF antagonist over a period of time of from about 2 to about 7 days, beginning on the day of menses, to 5 days following the start of menses, or 1-5 days following the start of menses (e.g., days 1-5 of the menstrual cycle); and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg VEGF antagonist over a period of time of from about 2 to about 7 days beginning on the day of menses to 5 days following the start of menses (e.g., days 1-5 of the menstrual cycle), or 1-5 days following the start of menses; and administration of a progestin. In some of these embodiments, the methods further comprise administration of an estrogen.

In another particular embodiment, a subject combination treatment for inhibiting pregnancy comprises administering a dosage containing from about 0.05 mg/kg to about 20 mg/kg VEGF antagonist continuously or substantially continuously over a period of time of from about 1 day to about 14 days beginning on the day of menses to 5 days following the start of menses, or 1-5 days following the start of menses; and administration of a progestin. In some of these embodiments, the methods further comprise administration of an estrogen.

In a particular embodiment, a subject combination treatment for inhibiting pregnancy comprises subcutaneous administration by injection of from about 0.05 mg/kg to about 20 mg/kg chimeric VEGF blocker on about day 2 and about day 14 of the menstrual cycle; and administration of a progestin. In some of these embodiments, the methods further comprise administration of an estrogen.

In a particular embodiment, a subject combination treatment for inhibiting pregnancy comprises intravenous administration of from about 0.05 mg/kg to about 20 mg/kg chimeric VEGF blocker on about day 2 and about day 14 of the menstrual cycle; and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg chimeric VEGF blocker on about day 2 and about day 14 of the menstrual cycle; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg chimeric VEGF blocker on about day 2 and about day 14 of the menstrual cycle; and administration of a progestin. In some of these embodiments, the methods further comprise administration of an estrogen.

In a particular embodiment, a subject combination treatment for inhibiting pregnancy comprises subcutaneous administration by injection of from about 0.05 mg/kg to about 20 mg/kg chimeric VEGF blocker on about day 1 and about day 14 of the menstrual cycle; and administration of a progestin. In some of these embodiments, the methods further comprise administration of an estrogen.

In a particular embodiment, a subject combination treatment for inhibiting pregnancy comprises intravenous administration of from about 0.05 mg/kg to about 20 mg/kg chimeric VEGF blocker on about day 1 and about day 14 of the menstrual cycle; and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg chimeric VEGF blocker on about day 1 and about day 14 of the menstrual cycle; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg chimeric VEGF blocker on about day 1 and about day 14 of the menstrual cycle; and administration of a progestin. In some of these embodiments, the methods further comprise administration of an estrogen.

In a particular embodiment, a subject combination treatment for inhibiting pregnancy comprises subcutaneous administration by injection-of from about 0.05 mg/kg to about 20 mg/kg chimeric VEGF blocker on about day 2 and about day 4 of the menstrual cycle; and administration of a progestin. In some of these embodiments, the methods further comprise administration of an estrogen.

In a particular embodiment, a subject combination treatment for inhibiting pregnancy comprises intramuscular administration by injection of from about 0.05 mg/kg to about 20 mg/kg chimeric VEGF blocker on about day 2 and about day 4 of the menstrual cycle; and administration of a progestin. In some of these embodiments, the methods further comprise administration of an estrogen.

In a particular embodiment, a subject combination treatment for inhibiting pregnancy comprises intravenous administration of from about 0.05 mg/kg to about 20 mg/kg chimeric VEGF blocker on about day 2 and about day 4 of the menstrual cycle;

and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg chimeric VEGF blocker on about day 2 and about day 4 of the menstrual cycle; and administration of a progestin. In some of these embodiments, the methods further comprise administration of an estrogen.

In a particular embodiment, a subject combination treatment for inhibiting pregnancy comprises subcutaneous administration by injection of from about 0.05 mg/kg to about 20 mg/kg chimeric VEGF blocker over a period of time of from about 2 to about 7 days beginning on the day of menses to 5 days following the start of menses, or 1-5 days following the start of menses; and administration of a progestin. In some of these embodiments, the methods further comprise administration of an estrogen.

In another particular embodiment, a subject combination treatment for inhibiting pregnancy comprises intramuscular administration by injection of from about 0.05 mg/kg to about 20 mg/kg chimeric VEGF blocker over a period of time of from about 2 to about 7 days beginning on the day of menses to 5 days following the start of menses, or 1-5 days following the start of menses; and administration of a progestin. In some of these embodiments, the methods further comprise administration of an estrogen.

In a particular embodiment, a subject combination treatment for inhibiting pregnancy comprises intravenous administration of from about 0.05 mg/kg to about 20 mg/kg chimeric VEGF blocker over a period of time of from about 2 to about 7 days beginning on the day of menses to 5 days following the start of menses, or 1-5 days following the start of menses (e.g., days 1-5 of the menstrual cycle); and administration of a progestin. In some of these embodiments, the methods further comprise administration of an estrogen.

In a particular embodiment, a subject combination treatment for inhibiting pregnancy comprises oral administration of from about 0.05 mg/kg to about 20 mg/kg chimeric VEGF blocker over a period of time of from about 2 to about 7 days beginning on the day of menses to 5 days following the start of menses, or 1-5 days following the start of menses; and administration of a progestin. In some of these embodiments, the methods further comprise administration of an estrogen.

In another particular embodiment, a subject combination treatment for inhibiting pregnancy comprises administering a dosage containing from about 0.05 mg/kg to about 20 mg/kg chimeric VEGF blocker continuously or substantially continuously over a period of time of from about 1 day to about 14 days beginning on the day of menses to 5 days following the start of menses, or 1-5 days following the start of menses; and administration of a progestin. In some of these embodiments, the methods further comprise administration of an estrogen.

In another particular embodiment, a subject combination treatment for inhibiting pregnancy comprises administering a dosage containing from about 0.05 mg/kg to about 20 mg/kg chimeric VEGF blocker continuously or substantially continuously over a period of time of from about 1 day to about 14 days beginning on the first day of menses; and administration of a progestin. In some of these embodiments, the methods further comprise administration of an estrogen.

In another particular embodiment, a subject combination treatment for inhibiting pregnancy comprises transvaginal administration of a chimeric VEGF blocker 3-5 days following the start of menses; and administration of a progestin. In some of these embodiments, the methods further comprise administration of an estrogen.

In another particular embodiment, a subject combination treatment for inhibiting pregnancy comprises transvaginal administration of a chimeric VEGF blocker for a period of time of from the first day of menses to about 14 days, or about 1 day to about 14 days beginning on the first day of menses; and administration of a progestin. In some of these embodiments, the methods further comprise administration of an estrogen.

In another particular embodiment, a subject combination treatment for inhibiting pregnancy comprises transvaginal administration of a chimeric VEGF blocker for a period of time of from about 1 day to about 10 days beginning on day 3 of the menstrual cycle; and administration of a progestin. In some of these embodiments, the methods further comprise administration of an estrogen.

In another particular embodiment, a subject combination treatment for inhibiting pregnancy comprises transvaginal administration of a chimeric VEGF blocker for a period of time of from about 1 day to about 10 days beginning on day 4 of the menstrual cycle; and administration of a progestin. In some of these embodiments, the methods further comprise administration of an estrogen.

In another particular embodiment, a subject combination treatment for inhibiting pregnancy comprises transvaginal administration of a chimeric VEGF blocker for a period of time of from about 1 day to about 10 days beginning on day 5 of the menstrual cycle; and administration of a progestin. In some of these embodiments, the methods further comprise administration of an estrogen.

Combination Therapy for Treating Abnormal Gynecological Bleeding

In some embodiments, a VEGF antagonist (e.g., a chimeric VEGF blocker) is administered with a second therapeutic agent that treats abnormal gynecological bleeding. Suitable second therapeutic agents that treat dysfunctional-gynecological bleeding include, but are not limited to, a gestagen;-large-dose estrogen/gestagen combinations; a nonsteroidal cyclooxygenase inhibitor; and a luteinizing hormone releasing hormone (LHRH) agonist.

Any of the above-described treatment regimens comprising administering a VEGF antagonist (e.g., a chimeric VEGF blocker) can be modified to include administration of a second therapeutic agent to treat abnormal gynecological bleeding.

Suitable second therapeutic agents that treat dysfunctional gynecological bleeding include, but are not limited to, a gestagen (e.g., 10 mg of medroxyprogesterone acetate daily, or 0.7-1.0 mg of norethindrone acetate daily, each for 10-14 days); large-dose estrogen/gestagen combinations over a period of 10-14 days; a nonsteroidal cyclooxygenase inhibitor (e.g., mefenamic acid, naproxen, ibuprofen); and an LHRH agonist.

In some embodiments, for the treatment of dysfunctional gynecological bleeding, a combination therapy comprising administration of a VEGF antagonist (e.g., a chimeric VEGF blocker) and a second therapeutic agent is carried out.

In some embodiments, a VEGF antagonist is administered in conjunction with an agent that regulates menses. In some of these embodiments, an agent that regulates menses is administered for a first period of time, during which menses does not occur; and, after the first period of time, the agent is withdrawn (i.e., not administered to the individual). Menses occurs following withdrawal (absence of administration) of the agent. A VEGF antagonist is then administered before the mid-proliferative phase, e.g., before day 10 of the menstrual cycle, as described above. Suitable treatment regimens to induce withdrawal bleeding include, but are not limited to, 1) medroxyprogesterone acetate (Provera®), 5 mg twice a day, or 10 mg once a day, for 5 days; 2) micronized progesterone, 200 mg to 300 mg daily for 10 days; 3) progesterone in oil, administered by intramuscular injection, 100 mg to 200 mg in one dose; 4) oral contraceptive pills (most monophasic pills with all the same dose of estrogen and progestogen), one pill each day for 4 to 5 days. In some cases, e.g., where abnormal gynecological bleeding has been very heavy or prolonged, the progestogen will be administered for longer than 5 days, or longer than 10 days.

The following are non-limiting examples.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by oral administration of a VEGF antagonist containing an amount of 0.05 mg/kg to about 20 mg/kg VEGF antagonist day 1 or day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intramuscular injection, a VEGF antagonist containing an amount of 0.05 mg/kg to about 20 mg/kg VEGF antagonist day 1 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by subcutaneous injection, a VEGF antagonist containing an amount of 0.05 mg/kg to about 20 mg/kg VEGF antagonist day 1 or day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intravenous injection, a VEGF antagonist containing an amount of 0.05 mg/kg to about 20 mg/kg VEGF antagonist day 1 or day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intramuscular injection, a VEGF antagonist containing an amount of 0.05 mg/kg to about 20 mg/kg VEGF antagonist day 1 or day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering orally a VEGF antagonist containing an amount of 0.05 mg/kg to about 20 mg/kg VEGF antagonist 2 days of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by subcutaneous injection, a VEGF antagonist containing an amount of 0.05 mg/kg to about 20 mg/kg VEGF antagonist on day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intravenous injection, a VEGF antagonist containing an amount of 0.05 mg/kg to about 20 mg/kg VEGF antagonist on day 2 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intramuscular injection, a VEGF antagonist containing an amount of 0.05 mg/kg to about 20 mg/kg VEGF antagonist on day 3 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering orally a VEGF antagonist containing an amount of 0.05 mg/kg to about 20 mg/kg VEGF antagonist on day 3 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by subcutaneous injection, a VEGF antagonist containing an amount of 0.05 mg/kg to about 20 mg/kg VEGF antagonist on day 3 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intravenous injection, a VEGF antagonist containing an amount of 0.05 mg/kg to about 20 mg/kg VEGF antagonist on day 3 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intramuscular injection, a VEGF antagonist containing an amount of 0.05 mg/kg to about 20 mg/kg VEGF antagonist on day 2 and on day 14 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering orally a VEGF antagonist containing an amount of 0.05 mg/kg to about 20 mg/kg VEGF antagonist on day 2 and on day 14 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by subcutaneous injection, a VEGF antagonist containing an amount of 0.05 mg/kg to about 20 mg/kg VEGF antagonist on day 2 and on day 14 of the menstrual cycle.

In particular embodiments, a subject combination therapy for treating abnormal gynecological bleeding comprises administering an amount of medroxyprogesterone acetate (Provera®) containing 5 mg twice a day administered orally, or 10 mg once a day administered orally, for 5 days; followed by administering, by intravenous injection, a VEGF antagonist containing an amount of 0.05 mg/kg to about 20 mg/kg VEGF antagonist on day 2 and on day 14 of the menstrual cycle.

In particular embodiments, a VEGF antagonist (e.g., a soluble VEGF receptor) is administered in combination therapy with a gestagen. In some of these embodiments, a subject combination therapy for treating abnormal gynecological bleeding comprises administration of 10 mg of medroxyprogesterone acetate daily or 0.7-1.0 mg of norethindrone acetate daily, each for 10-14 days; and subcutaneous administration by injection of from about 0.05 mg/kg to about 20 mg/kg chimeric VEGF blocker over a period of time from about 2 to about 7 days beginning 1-5 days, 2-5 days, or 3-5 days following the start of menses (e.g., beginning on day 1 to day 5, beginning on day 2 to day 5, or beginning on day 3 to day 5 of the menstrual cycle).

In other embodiments, a subject combination therapy comprises administration of 10 mg of medroxyprogesterone acetate daily or 0.7-1.0 mg of norethindrone acetate daily, each for 10-14 days; and a single subcutaneous administration by injection of from about 0.05 mg/kg to about 5 mg/kg soluble VEGF receptor 1-5 days, 2-5 days, or 3-5 days following the start of menses. In other embodiments, a subject combination therapy comprises administration of 10 mg of medroxyprogesterone acetate daily or 0.7-1.0 mg of norethindrone acetate daily, each for 10-14 days; and intramuscular administration by injection of from about 0.05 mg/kg to about 10 mg/kg chimeric VEGF blocker over a period of time from about 2 to about 7 days 1-5 days, 2-5 days, or beginning 3-5 days following the start of menses. In other embodiments, a subject combination therapy comprises administration of 10 mg of medroxyprogesterone acetate daily or 0.7-1.0 mg of norethindrone acetate daily, each for 10-14 days; and a single intramuscular injection of from about 0.05 mg/kg to about 5 mg/kg chimeric VEGF blocker 1-5 days, 2-5 days, or 3-5 days following the start of menses. In other embodiments, a subject combination therapy comprises administration of 10 mg of medroxyprogesterone acetate daily or 0.7-1.0 mg of norethindrone acetate daily, each for 10-14 days; and transvaginal administration of a chimeric VEGF blocker 1-5 days, 2-5 days, or 3-5 days following the start of menses.

In other particular embodiments, a chimeric VEGF blocker is administered in combination therapy with large-dose estrogen/gestagen combinations over a period of 10-14 days. In some of these embodiments, a subject combination therapy comprises administration of large-dose estrogen/gestagen combinations over a period of 10-14 days; and subcutaneous administration by injection of from about 0.05 mg/kg to about 10 mg/kg chimeric VEGF blocker over a period of time from about 2 to about 7 days beginning 1-5 days, 2-5 days, or 3-5 days following the start of menses. In other embodiments, a subject combination therapy comprises administration of large-dose estrogen/gestagen combinations over a period of 10-14 days; and a single subcutaneous administration by injection of from about 0.05 mg/kg to about 5 mg/kg chimeric VEGF blocker 1-5 days, 2-5 days, or 3-5 days following the start of menses. In other embodiments, a subject combination therapy comprises administration of large-dose estrogen/gestagen combinations over a period of 10-14 days; and intramuscular administration by injection of from about 0.05 mg/kg to about 10 mg/kg chimeric VEGF blocker over a period of time from about 2 to about 7 days beginning 1-5 days, 2-5 days, or 3-5 days following the start of menses. In other embodiments, a subject combination therapy comprises administration of large-dose estrogen/gestagen combinations over a period of 10-14 days; and a single intramuscular injection of from about 0.05 mg/kg to about 5 mg/kg chimeric VEGF blocker 1-5 days, 2-5 days, or 3-5 days following the start of menses. In other embodiments, a subject combination therapy comprises administration of large-dose estrogen/gestagen combinations over a period of 10-14 days; and transvaginal administration of a chimeric VEGF blocker 1-5 days, 2-5 days, or 3-5 days following the start of menses.

In particular embodiments, a chimeric VEGF blocker is administered in combination therapy with a gestagen. In some of these embodiments, a subject combination therapy comprises administration of a nonsteroidal cyclooxygenase inhibitor; and subcutaneous administration by injection of from about 0.05 mg/kg to about 10 mg/kg chimeric VEGF blocker over a period of time from about 2 to about 7 days beginning 1-5 days, 2-5 days, or 3-5 days following the start of menses. In other embodiments, a subject combination therapy comprises administration of a nonsteroidal cyclooxygenase inhibitor; and a single subcutaneous administration by injection of from about 0.05 mg/kg to about 5 mg/kg chimeric VEGF blocker 1-5 days, 2-5 days, or 3-5 days following the start of menses. In other embodiments, a subject combination therapy comprises administration of a nonsteroidal cyclooxygenase inhibitor; and intramuscular administration by injection of from about 0.05 mg/kg to about 10 mg/kg chimeric VEGF blocker over a period of time from about 2 to about 7 days beginning 1-5 days, 2-5 days, or 3-5 days following the start of menses. In other embodiments, a subject combination therapy comprises administration of a nonsteroidal cyclooxygenase inhibitor; and a single intramuscular injection of from about 0.05 mg/kg to about 5 mg/kg chimeric VEGF blocker 1-5 days, 2-5 days, or 3-5 days following the start of menses. In other embodiments, a subject combination therapy comprises administration of a nonsteroidal cyclooxygenase inhibitor; and transvaginal administration of a chimeric VEGF blocker 1-5 days, 2-5 days, or 3-5 days following the start of menses.

In particular embodiments, a soluble VEGF receptor is administered in combination therapy with a gestagen. In some of these embodiments, a subject combination therapy comprises administration of 10 mg of medroxyprogesterone acetate daily or 0.7-1.0 mg of norethindrone acetate daily, each for 10-14 days; and

subcutaneous administration by injection of from about 0.05 mg/kg to about 20 mg/kg chimeric VEGF blocker on about day 2 and about day 14 of the menstrual cycle.

In other embodiments, a subject combination therapy comprises administration of 10 mg of medroxyprogesterone acetate daily or 0.7-1.0 mg of norethindrone acetate daily, each for 10-14 days; and subcutaneous administration by injection of from about 0.05 mg/kg to about 20 mg/kg chimeric VEGF blocker on about day 1 and about day 14 of the menstrual cycle.

In other embodiments, a subject combination therapy comprises administration of 10 mg of medroxyprogesterone acetate daily or 0.7-1.0 mg of norethindrone acetate daily, each for 10-14 days; and intramuscular administration by injection of from about 0.05 mg/kg to about 20 mg/kg chimeric VEGF blocker on about day 3 and about day 14 of the menstrual cycle. In other embodiments, a subject combination therapy comprises administration of 10 mg of medroxyprogesterone acetate daily or 0.7-1.0 mg of norethindrone acetate daily, each for 10-14 days; and a single intramuscular injection of from about 0.05 mg/kg to about 20 mg/kg chimeric VEGF blocker on about day 4 and about day 14 of the menstrual cycle.

In other embodiments, a subject combination therapy comprises administration of 10 mg of medroxyprogesterone acetate daily or 0.7-1.0 mg of norethindrone acetate daily, each for 10-14 days; and transvaginal administration of a chimeric VEGF blocker from about day 3 to day 5, from about day 1 to day 7, from about day 7 to day 14, from about day 1 to day 14, from about day 2 to day 7, or from about day 2 to day 14 of the menstrual cycle.

Further Combinations

In some embodiments, a VEGF antagonist (e.g., a chimeric VEGF blocker) is administered with a second therapeutic agent that treats an infection, e.g., a yeast infection, such as a vaginal yeast infection. Suitable second therapeutic agents that treat yeast infections include, but are not limited to, miconazole nitrate (Monistat®), butoconazole nitrate (Femstat3®), and clotrimazole (Gyne-Lotrimin®). Furthermore, any of the above-described therapeutic regimens to inhibit pregnancy or to treat abnormal gynecological bleeding can be modified to include administering an agent that treats an infection, e.g., a yeast infection.

Use of VEGF Antagonists

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage units for day 1 of the menstrual cycle comprises an amount of a VEGF antagonist effective to inhibit vascular proliferation in the endometrium of the uterus of the human, and where each of the dosage units for the remaining 27 days contain placebo (i.e., each of the dosage units for the remaining 27 days do not contain the VEGF antagonist.

A typical menstrual cycle is 28 days, although a normal menstrual cycle can vary from 21 days to 35 days. Thus, the term, “menstrual cycle,” as used herein, includes normal variations from the 28 day cycle, e.g., 27 days, 30 days, etc. Accordingly, those skilled in the art will recognize that the descriptions of a preparation comprising a packaging unit for oral administration for a period of 28 consecutive days can be modified to accommodate normal variations in menstrual cycles.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage unit for day 2 of the menstrual cycle comprises an amount of a VEGF antagonist effective to inhibit vascular proliferation in the endometrium of the uterus of the human, and where each of the dosage units for the remaining 27 days contain placebo (i.e., each of the dosage units for the remaining 27 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage unit for day 3 of the menstrual cycle comprises an amount of a VEGF antagonist effective to inhibit vascular proliferation in the endometrium of the uterus of the human, and where each of the dosage units for the remaining 27 days contain placebo (i.e., each of the dosage units for the remaining 27 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage unit for day 4 of the menstrual cycle comprises an amount of a VEGF antagonist effective to inhibit vascular proliferation in the endometrium of the uterus of the human, and where each of the dosage units for the remaining 27 days contain placebo (i.e., each of the dosage units for the remaining 27 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage unit for day 5 of the menstrual cycle comprises an amount of a VEGF antagonist effective to inhibit vascular proliferation in the endometrium of the uterus of the human, and where each of the dosage units for the remaining 27 days contain placebo (i.e., each of the dosage units for the remaining 27 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage units for days 2 and 14 of the menstrual cycle comprise an amount of a VEGF antagonist effective to inhibit vascular proliferation in the endometrium of the uterus of the human, and where each of the dosage units for the remaining 26 days contain placebo (i.e., each of the dosage units for the remaining 26 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage units for days 3 and 14 of the menstrual cycle comprise an amount of a VEGF antagonist effective to inhibit vascular proliferation in the endometrium of the uterus of the human, and where each of the dosage units for the remaining 26 days contain placebo (i.e., each of the dosage units for the remaining 26 days do not contain the VEGF antagonist.

The present invention further provides use of a VEGF antagonist in the preparation of a pharmaceutical preparation for inhibiting implantation of a blastocyst in the uterine endometrium of a female human of child-bearing age, wherein the preparation is in the form of separately packaged and individually removable daily dosage units arranged in a packaging unit and intended for oral administration for a period of 28 consecutive days; wherein the daily dosage units for days 1 and 14 of the menstrual cycle comprise an amount of a VEGF antagonist effective to inhibit vascular proliferation in the endometrium of the uterus of the human, and where each of the dosage units for the remaining 26 days contain placebo (i.e., each of the dosage units for the remaining 26 days do not contain the VEGF antagonist.

Subjects Suitable for Treatment

Subjects suitable for treatment with a subject method for inhibiting pregnancy include female humans of childbearing age. Subjects suitable for treatment with a subject method for treating abnormal gynecological bleeding include female humans of childbearing age; pre-menopausal women; post-menopausal women; perimenopausal women; and adolescent females.

Women who experience dysfunctional gynecological bleeding are suitable for treatment with a subject method, e.g., premenopausal women; perimenopausal women; adolescents; women experiencing postmenopausal bleeding; women experiencing breakthrough bleeding; women with endometrial hyperplasia; women with endometrial polyps; and women who are taking an oral contraceptive and who are experiencing abnormal bleeding as a result of the oral contraceptive.

Women who are at a stage in the menstrual cycle that is one or after the beginning of menses, but before the mid-proliferative phase (e.g., before day 10 of the menstrual cycle) are suitable for treatment with a subject method. For example, women at day 1 of the menstrual cycle, women at day 2 of the menstrual cycle, women at day 3 of the menstrual cycle, women at day 4 of the menstrual cycle, women at day 5 of the menstrual cycle, women at day 6 of the menstrual cycle, and women at day 7 of the menstrual cycle, are suitable for treatment with a subject method. In many embodiments, the woman has not been treated with a VEGF antagonist within 5 days to about 27 days prior to the dosing event with the VEGF antagonist.

Formulations, Delivery Systems, and Kits

The present invention further provides chimeric VEGF blockers formulated for use in inhibiting pregnancy and/or treating gynecological bleeding disorders, as well as kits comprising the formulations. The present invention further provides delivery systems comprising a subject formulation, for delivery of a subject formulation to a human female subject in need thereof.

Formulations

The present invention provides formulations comprising a chimeric VEGF blocker. In some embodiments, a subject formulation is suitable for systemic delivery, e.g., by injection. In other embodiments, a subject formulation is suitable for intravaginal delivery. In other embodiments, a subject formulation is suitable for transdermal delivery. In other embodiments, a subject formulation is suitable for intrauterine delivery.

In many embodiments, a subject formulation comprises from about 0.01 mg to about 50 mg per unit formulation (e.g., per ml liquid formulation or per gram gel, solid or semi-solid formulation), e.g., from about 0.01 mg to about 0.05 mg, from about 0.05 mg to about 0.1 mg, from about 0.1 mg to about 0.5 mg, from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, from about 4.5 mg to about 5.0 mg, from about 5.0 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, or from about 45 mg to about 50 mg chimeric VEGF blocker.

A subject formulation for intravaginal administration is formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

A subject formulation includes one or more of an excipient (e.g., sucrose, starch, mannitol, sorbitol, lactose, glucose, cellulose, talc, calcium -phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Chimeric VEGF blocker formulations suitable for injection include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacterial and fungi. The carrier can be a solvent or dispersion medium containing, for example, water, ethanol (e.g., glycerol, propylene glycol and liquid polyethylene glycol), suitable mixtures thereof, and vegetable oil.

Tablets comprising a chimeric VEGF blocker may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and

the like.

Delivery Systems

The present invention provides a delivery system comprising a subject chimeric VEGF blocker formulation. In some embodiments, the delivery system is a delivery system that provides for injection of a subject formulation subcutaneously or intramuscularly. In other embodiments, the delivery system is a vaginal delivery system. In other embodiments, the delivery system is an intrauterine delivery system. In some embodiments, the delivery system includes a device that is an implant, e.g., an intrauterine device, an intracervical device, or an intravaginal device.

Injection Systems

In some embodiments, a subject delivery system comprises an injection device, Exemplary, non-limiting drug delivery devices include injections devices, such as pen injectors, and needle/syringe devices. In some embodiments, the invention provides an injection delivery device that is pre-loaded with a formulation comprising an effective amount of a chimeric VEGF blocker. For example, a subject delivery device comprises an injection device pre-loaded with a single dose of a chimeric VEGF blocker. A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

Vaginal Drug Delivery Systems

The present invention provides a vaginal delivery system for transvaginal delivery of a chimeric VEGF blocker (“drug”) to the endometrium of an individual. The delivery system comprises a device for insertion into the vagina. In some embodiments, the delivery system comprises an applicator for delivery of a formulation into the vagina;

and a container that contains a formulation comprising a chimeric VEGF blocker. In these embodiments, the container (e.g., a tube) is adapted for delivering a formulation into the applicator. In other embodiments, the delivery system comprises a device that is inserted into the vagina, which device includes a chimeric VEGF blocker. For example, the device is coated with, impregnated with, or otherwise contains a formulation comprising a chimeric VEGF blocker.

In some embodiments, the vaginal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909 If a tampon or tampon-like device is used, there are numerous methods by which a drug (e.g., a chimeric VEGF blocker) can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon, for example, by dissolving the drug in a pharmaceutically acceptable carrier and absorbing the drug solution into the tampon fibers. The drug can also be dissolved in a coating material which is applied to the tip of the tampon. Alternatively, the drug can be incorporated into an insertable suppository which is placed in association with the tip of the tampon.

In other embodiments, the drug delivery device is a vaginal ring. Vaginal rings usually consist of an inert elastomer ring coated by another layer of elastomer containing the drug (e.g., chimeric VEGF blocker) to be delivered. The rings can be easily inserted, left in place for the desired period of time (e.g., up to 7 days), then removed by the user. The ring can optionally include a third, outer, rate-controlling elastomer layer which contains no drug. Optionally, the third ring can contain a second drug for a dual release ring. The drug can be incorporated into polyethylene glycol -throughout the silicone elastomer ring to act as a reservoir for drug to be delivered.

In other embodiments, a subject vaginal delivery system is a vaginal sponge. The chimeric VEGF blocker is incorporated into a silicone matrix which is coated onto a cylindrical drug-free polyurethane vaginal sponge, as described in the literature.

Pessaries, tablets and suppositories are other examples of drug delivery systems which can be used in the present invention. These systems have been used for delivery of vaginal medications and steroids, and have been described extensively in the literature.

Bioadhesive microparticles constitute still another drug delivery system suitable for use in the present invention. This system is a multi-phase liquid or semi-solid preparation which does not seep from the vagina as do most current suppository formulations. The substances cling to the wall of the vagina and release the drug (e.g., chimeric VEGF blocker) over a several hour period of time. Many of these systems were designed for nasal use but can be used in the vagina as well (e.g. U.S. Pat. No. 4,756,907). The system may comprise microspheres with an active drug (e.g., soluble VEGF receptor) and a surfactant for enhancing uptake of the drug. The microparticles have a diameter of 10-100 pm and can be prepared from starch, gelatin, albumin, collagen, or dextran.

Another system is a container comprising a subject formulation (e.g., a tube) that is adapted for use with an applicator. The chimeric VEGF blocker polypeptide is incorporated into creams, lotions, foams, paste, ointments, and gels which can be applied to the vagina using an applicator. Processes for preparing pharmaceuticals in cream, lotion, foam, paste, ointment and gel formats can be found throughout the literature. An example of a suitable system is a standard -fragrance free lotion formulation containing glycerol, ceramides, mineral oil, petrolatum, parabens, fragrance and water such as the product sold under the trademark JERGENS™ (Andrew Jergens Co., Cincinnati, Ohio). This formulation was used by Hargrove et al. (Abstract No. 97.051, North American Menopause Society, Boston, Mass., September, 1997) for transcutaneous delivery of estradiol and progesterone. Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal dosage form desired, e.g., whether the active ingredient(s) is/are to be formulated into a cream, lotion, foam, ointment, paste, solution, or gel, as well as on the identity of the active ingredient(s).

Other suitable delivery devices are those described in U.S. Pat. No. 6,476,079.

Intrauterine Delivery Systems

In some embodiments, a subject delivery system for delivering a VEGF blocker to an individual is an intrauterine delivery system, comprising an intrauterine device that contains, is coated with, is impregnated with, or is otherwise adapted for delivery of, a VEGF blocker. Intrauterine devices (IUD) are well known in the art, and any known IUD can be used in a subject delivery system. See, e.g., Andersson et al. (1992) Obstet. Gynecol. 79:963; and U.S. Pat. No. 3,967,618. A suitable IUD includes an intrauterine device as described in U.S. Pat. No. 6,476,079.

Kits

The invention further provides kits for practicing the methods of the invention, where the kit comprises a formulation and/or a delivery system comprising a VEGF antagonist. In some embodiments, a subject kit includes an injection delivery device preloaded with a VEGF antagonist formulation. In other embodiments, a subject kit comprises a subject vaginal drug delivery device that comprises a VEGF antagonist formulation.

In many embodiments of the subject kits, the kits will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the VEGF antagonist at the appropriate time during the menstrual cycle. Such components include, but are not limited to, a calendaring system to aid the patient in remembering to take the VEGF antagonist at the appropriate time during the menstrual cycle.

A typical menstrual cycle is 28 days, although a normal menstrual cycle can vary from 21 days to 35 days. Thus, the term, “menstrual cycle,” as used herein, includes normal variations from the 28 day cycle, e.g., 27 days, 30 days, etc. Those skilled in the art will recognize that the descriptions of a kit, e.g., comprising a package containing spaces for 28 tablets can be modified to accommodate normal variations in menstrual cycles, such that, e.g., a package could contain spaces for only 21 tablets.

In some embodiments, e.g., where oral delivery is the route of administration, a subject kit includes a package containing spaces for 28 tablets is provided, including spaces for placebo (formulation without VEGF antagonist) for days on which VEGF antagonist is not administered, is provided. In other embodiments, e.g., where the route of delivery is intravaginal, a subject kit includes a package containing suppositories, vaginal inserts (e.g., tampons), or other intravaginal delivery systems, is provided with placebo (delivery device without VEGF antagonist) for days on which VEGF antagonist is not administered, is provided. For example, a subject kit includes a package that contains, in spaces for days 1 and/or 2 and/or 3 and/or 4 and/or 5, a tablet, tampon, suppository, or other formulation or delivery means, that comprises a VEGF antagonist; and in spaces for all other days of a 28-day cycle, contains placebo.

As another example, a subject kit includes a package that contains, in spaces for days 1-14 of the menstrual cycle, a tablet, tampon, suppository, or other formulation-or delivery means, that comprises a VEGF antagonist; and in spaces for all other days of a 28-day cycle, contains placebo. As another example, a subject kit includes a package that contains, in spaces for day 2 and day 14 of the menstrual cycle, a tablet, tampon, suppository, or other formulation or delivery means, that comprises a VEGF antagonist; and in spaces for all other days of a 28-day cycle, contains placebo. As another example, a subject kit includes a package that contains, in spaces for day 3 and day 14 of the menstrual cycle, a tablet, tampon, suppository, or other formulation or delivery means, that comprises a VEGF antagonist; and in spaces for all other days of a 28-day cycle, contains placebo.

The present invention provides a packaging unit comprising the daily dosage units described above, which may be prepared in a manner analogous to that of making other oral contraceptives. For example, the packaging unit is in some embodiments a conventional blister pack or any other form that it is typically used for oral contraceptive tablets, pills, and the like. The blister pack will contain the appropriate number of unit dosage forms (e.g., usually 28, or a multimer of 28), in a sealed blister pack with a cardboard, paperboard, foil, or plastic backing, and enclosed in a suitable cover. Each blister container may be numbered or otherwise labeled, e.g., starting with day 1, etc.

In some embodiments, the present invention provides a pharmaceutical preparation of a VEGF antagonist, the preparation comprising a number of separately packaged and individually removable daily dosage units arranged in a packaging unit, for oral administration for a period of 28 consecutive days, one dosage unit per day, where the daily dosage units for day 1 and day 2 of the menstrual cycle comprises an amount of a VEGF antagonist effective to inhibit endometrial vascular proliferation, and where the dosage units for the remaining 27 days contains placebo (i.e., the dosage units for the remaining 27 days do not contain the VEGF antagonist).

In some embodiments, the present invention provides a pharmaceutical preparation of a VEGF antagonist, the preparation comprising a number of separately packaged and individually removable daily dosage units arranged in a packaging unit, for oral administration for a period of 28 consecutive days, one dosage unit per day, where the daily dosage units for days 2, 4, and 6 of the menstrual cycle comprises an amount of a VEGF antagonist effective to inhibit endometrial vascular proliferation, and where the dosage units for the remaining 25 days contains placebo (i.e., the dosage units for the remaining 25 days do not contain the VEGF antagonist).

In some embodiments, the present invention provides a pharmaceutical preparation of a VEGF antagonist, the preparation comprising a number of separately packaged and individually removable daily dosage units arranged in a packaging unit, for oral administration for a period of 28 consecutive days, one dosage unit per day, where the daily dosage units for days 2 and 14 comprise an amount of a VEGF antagonist effective to inhibit endometrial vascular proliferation, and where the dosage units for the remaining 26 days contains placebo (i.e., the dosage units for the remaining 26 days do not contain the VEGF antagonist).

In any of the above-described embodiments, a subject kit may further comprise unit dosage forms of a progestogen, to promote menses. Such kits are useful for carrying out a subject method of treating abnormal gynecological bleeding. For example, the kit may comprise five separately packaged unit dosage forms (e.g., tablets) arranged in a packaging unit, each unit dosage form comprising medroxyprogesterone acetate (Provera®) 10 mg in five consecutive daily dosage units, labeled for oral ingestion of one unit dosage form per day for five days; and a number of separately packaged and individually removable daily dosage units arranged in a packaging unit, for oral administration for a period of 28 consecutive days, one dosage unit per day, where the daily. dosage units for days 2 and 14 comprise an amount of a VEGF antagonist effective to inhibit endometrial vascular proliferation, and where the dosage units for the remaining 26 days contains placebo (i.e., the dosage units for the remaining 26 days do not contain the VEGF antagonist). The kit will generally include written instructions to take one medroxyprogesterone tablet per day for five days, wait for menses to begin, then take one unit dosage form per day of the remaining 28 unit dosage forms for 28 days.

Kits Comprising Chimeric a VEGF Blocker

The invention further provides kits for practicing the methods of the invention, where the kit comprises a chimeric VEGF blocker formulation and/or a delivery system comprising a chimeric VEGF blocker. In some embodiments, a subject kit includes. an injection delivery device preloaded with a subject formulation. In other embodiments, a subject kit comprises a subject vaginal drug delivery device that comprises a subject formulation.

In many embodiments of the subject kits, the kits will further include instructions for practicing the subject methods, or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the VEGF blocker at the appropriate time during the menstrual cycle. Such components include, but are not limited to, a calendaring system to aid the patient in remembering to take the VEGF blocker at the appropriate time during the menstrual cycle.

In some embodiments, e.g., where oral delivery is the route of administration, a subject kit includes a package containing spaces for 28 tablets is provided, including spaces for placebo (formulation without VEGF blocker) for days on which VEGF blocker is not administered, is provided. In other embodiments, e.g., where the route of delivery is intravaginal, a subject kit includes a package containing suppositories, vaginal inserts (e.g., tampons), or other intravaginal delivery systems, is provided with placebo (delivery device without VEGF blocker) for days on which VEGF blocker is not administered, is provided. For example, a subject kit includes a package that contains, in spaces for days 1 and/or 2 and/or 3 and/or 4 and/or 5, a tablet, tampon, suppository, or other formulation or delivery means, that comprises a VEGF blocker; and in spaces for all other days of a 28-day cycle, contains placebo.

As another example, a subject kit includes a package that contains, in spaces for days 1-14 of the menstrual cycle, a tablet, tampon, suppository, or other formulation or delivery means, that comprises a VEGF blocker; and in spaces for all other days of a 28-day cycle, contains placebo. As another example, a subject kit includes a package that contains, in spaces for day 2 and day 14 of the menstrual cycle, a tablet, tampon, suppository, or other formulation or delivery means, that comprises a VEGF blocker; and in spaces for all other days of a 28-day cycle, contains placebo. As another example, a subject kit includes a package that contains, in spaces for day 3 and day 14 of the menstrual cycle, a tablet, tampon, suppository, or other formulation or delivery means, that comprises a VEGF blocker; and in spaces for all other days of a 28-day cycle, contains placebo.

The present invention provides a packaging unit comprising the daily dosage units described above, which may be prepared in a manner analogous to that of making other oral contraceptives. For example, the packaging unit is in some embodiments a conventional blister pack or any other form that it is typically used for oral contraceptive tablets, pills, and the like. The blister pack will contain the appropriate number of unit dosage forms (e.g., usually 28, or a multimer of 28), in a sealed blister pack with a cardboard, paperboard, foil, or plastic backing, and enclosed in a suitable cover. Each blister container may be numbered or otherwise labeled, e.g., starting with day 1, etc.

In some embodiments, the present invention provides a pharmaceutical preparation of a chimeric VEGF blocker, the preparation comprising a number of separately packaged and individually removable daily dosage units arranged in a packaging unit, for oral administration for a period of 28 consecutive days, one dosage unit per day, where the daily dosage units for day 2 of the menstrual cycle comprises an amount of a chimeric VEGF blocker effective to inhibit endometrial vascular proliferation, and where the dosage units for the remaining 27 days contains placebo (i.e., the dosage units for the remaining 27 days do not contain the chimeric VEGF blocker).

In some embodiments, the present invention provides a pharmaceutical preparation of a chimeric VEGF blocker, the preparation comprising a number of separately packaged and individually removable daily dosage units arranged in a packaging unit, for oral administration for a period of 28 consecutive days, one dosage unit per day, where the daily dosage units for days 2, 4, and 6 of the menstrual cycle comprises an amount of a chimeric VEGF blocker effective to inhibit endometrial vascular proliferation, and where the dosage units for the remaining 25 days contains placebo (i.e., the dosage units for the remaining 25 days do not contain the chimeric VEGF blocker).

In some embodiments, the present invention provides a pharmaceutical preparation of a chimeric VEGF blocker, the preparation comprising a number of separately packaged and individually removable daily dosage units arranged in a packaging unit, for oral administration for a period of 28 consecutive days, one dosage unit per day, where the daily dosage units for days 2 and 14 comprise an amount of a chimeric VEGF blocker effective to inhibit endometrial vascular proliferation, and where the dosage units for the remaining 26 days contains placebo (i.e., the dosage units for the remaining 26 days do not contain the chimeric VEGF blocker).

### EXAMPLES

The following examples are put forth so as to provide those of ordinary skill in the art with a complete disclosure and description of how to make and use the present invention, and are not intended to limit the scope of what the inventor(s) regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Effect of VEGF Blockade on Primate Endometrium

In adult female primates, the endometrium undergoes shedding of the upper zones and cyclical repair and regeneration during the normal menstrual cycle. A key feature of this remarkable tissue remodeling is the growth of the vasculature. At the end of menstruation, the remaining deeper zones contain ruptured arterioles and venules. By day 5 after onset of menses, the blood vessels have healed and the surface epithelium has spread and covered the ragged surface. Subsequently there is a rapid growth of vasculature that supplies the upper layer of the regenerating endometrium. The growth. and development of blood vessels in the upper zones of endometrium during the postmenstrual phase play a critical role in subsequent growth and differentiation of the endometrium during later stages of the menstrual cycle.

The pattern of vascular endothelial growth factor (VEGF) expression and endothelial cell proliferation during the early- to mid-proliferative phase in the rhesus macaque endometrium shows the following properties: I) After menstrual sloughing of the upper zones (5-6 days after progesterone withdrawal), the newly formed surface epithelium showed a dramatic increase in VEGF mRNA expression during the postmenstrual repair stage. Also, there was significant increase in VEGF receptor Flt-1 and KDR mRNA expression in multiple profiles of small blood vessels just below the newly formed surface epithelium. In hormone deprived (HD) animals (after both estradiol and progesterone withdrawal), exactly the same pattern of up-regulation and localization of VEGF, Flt-1, and KDR mRNAs were found through HD 5-6 days in the surface epithelium and vascular endothelium, indicating that these up-regulation of VEGF and its receptors expression are independent of estradiol action. It may be noted that menstruation and healing of endometrium occur normally with or without the presence of estradiol after progesterone withdrawal. Several studies suggest a temporal and spatial correlation between the expression of VEGF and its receptors in cutaneous wound healing. For example, it has been shown that there is pronounced expression of VEGF in proliferating keratinocytes of the newly formed epithelium and heightened expression of Flt-1 and KDR in the capillary vessels in close vicinity to the epithelium during wound healing, which is consistent with the above-described observations during postmenstrual healing of endometrium, and indicates a role for VEGF in endometrial healing following menstruation.

Vascular endothelial proliferation in the endometrium showed a dramatic estrogen-dependent peak during the mid-proliferative phase. This proliferative peak coincided with a peak in VEGF expression in the endometrial stroma and VEGF expression in the stroma, not in the glands or surface epithelium, was significantly correlated with vascular proliferation throughout the menstrual cycle, suggesting roles for VEGF of stromal origin in endometrial vascular proliferation.

The following experiments demonstrate that blockade of VEGF action during the early- to mid-proliferative phase blocks endometrial healing and most vascular proliferation that occurs during the mid-proliferative phase.

To test the hypothesis that whether blockade of VEGF action during the postmenstrual phase can block most of the vascular growth and inhibit endometrial healing and regeneration, a compound called VEGF-Trap_{R1R2} (kegeneron Pharmaceuticals, Tarrytown, N.Y.), which is a soluble form of the VEGF receptor that can bind to VEGF, was used. VEGF-Trap_{R1R2} (“VEGF-Trap”) is a very potent inhibitor of VEGF action in vivo and in vitro. Holash et al. ((2002), supra). Six ovariectomized rhesus monkeys were used for this study: three controls and three treated animals (n=3). The animals were treated with VEGF-Trap at a dose rate of 12.5 mg/kg intravenously on days 2, 4, and 6, and the endometrium was collected on day 8, after progesterone withdrawal. The control animals were treated similarly with the vehicle. The results demonstrate that VEGF-trap blocks the E₂-dependent mid-proliferative peak in endometrial vascular proliferation (FIGS. 1A and 1B). There were several indications of delay in healing of endometrium after VEGF Trap treatment, including prolonged bleeding and focal hemorrhagic lesions on the surface epithelium, and inhibition of or atrophy of surface endothelium of the endometrium.

FIGS. 1A-D depict the effects of VEGF-trap in the rhesus macaque endometrium. FIGS. 1A and 1B depict inhibition of the mid-proliferative (8 days after progesterone withdrawal) vascular development and reepithelization of the rhesus macaque endometrium by blockade of VEGF action during the postmenstrual repair phase by VEGF-trap. FIGS. 1B and 1B depict hematoxilin and eosin staining of the endometrium. FIGS. 1C and 1D depict colocalization of BrdU and von Willebrand factor. In the control animals (A, C), the luminal epithelial cells (Le) are highly columnar (A) and show numerous Br-dU positive proliferating cells (C), and the upper endometrium shows large number of vonWillebrand factor stained vessels (C, arrows). In contrast, in VEGF-trap treated animals (B, D), the luminal epithelial cells are either atrophied or absent (B, arrow heads), the nuclei of very few cells show Br-dU immuno-staining (D), and the upper endometrial stroma is highly avascular with very few vonWillebrand factor stained blood vessels (D, arrow).

### Example 2

### Effect of VEGF Blockade on Endometrium in Primates

### Effects of Postmenstrual Administration of VEGF-Trap on Vascular Development and Progesterone-Induced Transformation of the Endometrium at the End of the Cycle

Studies were conducted in ovariectomized artificially cycling rhesus macaques. Following ovariectomy, the animals were treated sequentially with estradiol (E₂) for 14 days and with E₂ plus progesterone (P) for 14 days to create artificial menstrual cycles. At the end of the artificial cycle, P implants were removed to induce menstruation. The animals were treated with either a single dose intravenous administration of VEGF-trap (12.5 mg/kg body weight) on day 2 after P-withdrawal or with two doses of VEGF-trap, one on day 2 and the other on day 14 after P-withdrawal. The endometrium from these animals was collected on day 28 of the cycle (end of the cycle). In the previous study, 3 doses of VEGF-trap were used on days 2, 4, and 6 after P-withdrawal to block the midproliferative phase endometrial angiogenesis. In the study described in this example, different strategies and doses of VEGF-trap were used to block vascular development throughout the cycle.

VEGF-trap treatment (single dose) on day 2 after P-withdrawal resulted in about 24% reduction in vascular development in the upper endometrial zones. However, two doses of VEGF-trap, one on day 2 and the other on day 14 after P-withdrawal, caused a dramatic reduction (about 74%) in vascular density in the upper endometrial zones.

### Effects of Late-Proliferative Phase Administration of VEGF-Trap on Vascular Development and P-Induced Transformation of the Endometrium at the End of the Cycle

This experiment was performed identical to the above experiment, except that the VEGF-trap (single dose) was administered during the late-proliferative phase (days 10-14 after P withdrawal), and the endometrium from these animals was also collected on day 28 of the cycle (end of the cycle). As expected, no remarkable changes in vascular development in the upper endometrial zone were found in these animals.

These studies together clearly demonstrate that inhibition of endometrial vascular development in the upper endometrial zones can be achieved by blocking VEGF action (by VEGF-trap or any other VEGF antagonist) starting during the menstrual/postmenstrual period; continued treatment throughout the cycle is also efficacious, provided the treatment is initiated during the menstrual/postmenstrual period. Treatments initiated during later stages (after menstrual/postmenstrual period, e.g., late proliferative phase) of the cycle has no significant effect on endometrial vascular development.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inhibiting pregnancy in a female human, the method comprising administering to the female human during the postmenstrual repair phase a vascular endothelial growth factor receptor (VEGF) antagonist in an amount effective to inhibit vascular proliferation in the uterine endometrium.

2. The method of claim 1, wherein the VEGF antagonist is administered before day 10 of the menstrual cycle.

3. The method of claim 1, wherein the VEGF antagonist is administered during a period of time of between day 1 and day 7 of the menstrual cycle.

4. The method of claim 1, wherein the VEGF antagonist is administered on day 2 of the menstrual cycle.

5. The method of claim 1, wherein the VEGF antagonist is administered on day 2 and day 14 of the menstrual cycle.

6. The method of claim 1, wherein the VEGF antagonist is administered continuously or substantially continuously for a period of time of from about 1 day to about 14 days, beginning before day 10 of the menstrual cycle.

7. The method of claim 1, wherein the VEGF antagonist is administered transvaginally.

8. The method of claim 1, wherein the VEGF antagonist is administered by subcutaneous injection.

9. The method of claim 1, wherein the VEGF antagonist is administered by intramuscular injection.

10. The method of claim 1, wherein the VEGF antagonist is administered orally.

11. The method of claim 1, wherein the VEGF antagonist is a chimeric VEGF blocker polypeptide comprising, in order from amino terminus to carboxyl terminus, the second immunoglobulin domain of a VEGF receptor-1 polypeptide, the third immunoglobulin domain of a VEGF receptor-2 polypeptide, and an Fc portion of an immunoglobulin.

12. The method of claim 11, wherein the chimeric VEGF blocker polypeptide comprises an amino acid sequence as set forth in SEQ ID NO:16.

13. A method of treating abnormal gynecological bleeding in a female human, the method comprising administering to the female human during the postmenstrual repair phase a vascular endothelial growth factor receptor (VEGF) antagonist in an amount effective to inhibit vascular proliferation in the uterine endometrium.

14. The method of claim 13, wherein the VEGF antagonist is administered before day 10 of the menstrual cycle.

15. The method of claim 13, wherein the VEGF antagonist is administered during a period of time of between day 1 and day 7 of the menstrual cycle.

16. The method of claim 13, wherein the VEGF antagonist is administered on day 2 of the menstrual cycle.

17. The method of claim 13, wherein the VEGF antagonist is administered continuously or substantially continuously for a period of time of from about day 1 to about day 14 of the menstrual cycle.

18. The method of claim 13, wherein the VEGF antagonist is administered transvaginally.

19. The method of claim 13, wherein the VEGF antagonist is administered by subcutaneous injection.

20. The method of claim 13, wherein the VEGF antagonist is administered by intramuscular injection.

21. The method of claim 13, wherein the VEGF antagonist is administered orally.

22. The method of claim 13, wherein the method further comprises administering a progestogen for a period of time to induce menses, and wherein said progestogen administration is carried out before said VEGF antagonist administration.

23. The method of claim 13, wherein the VEGF antagonist is a chimeric VEGF blocker polypeptide comprising, in order from amino terminus to carboxyl terminus, the second immunoglobulin domain of a VEGF receptor-1 polypeptide, the third immunoglobulin domain of a VEGF receptor-2 polypeptide, and an Fc portion of an immunoglobulin.

24. The method of claim 23, wherein the chimeric VEGF blocker polypeptide comprises an amino acid sequence as set forth in SEQ ID-NO:16.

25. A pharmaceutical composition formulated for use in inhibiting pregnancy in a female human, the composition comprising:
a chimeric vascular endothelial cell receptor (VEGF) polypeptide, wherein the chimeric VEGF blocker polypeptide comprises, in order from amino terminus to carboxyl terminus, the second immunoglobulin domain of a VEGF receptor-1 polypeptide, the third immunoglobulin domain of a VEGF receptor-2 polypeptide, and an Fc portion of an immunoglobulin;
and a pharmaceutically acceptable excipient.

26. The composition of claim 25, wherein the chimeric VEGF blocker polypeptide is formulated as a bioadhesive microparticle, an intravaginal cream, an intravaginal lotion, an intravaginal foam, an intravaginal paste, an intravaginal ointment, an intravaginal solution or an intravaginal gel.

27. A drug delivery system for transvaginal delivery of a soluble vascular endothelial growth factor (VEGF) receptor polypeptide to the endometrium, the system comprising:
a pharmaceutical formulation comprising a chimeric VEGF blocker polypeptide, and
a delivery device adapted for delivery into the vagina.

28. The drug delivery system of claim 27, wherein said device is selected from a tampon, a vaginal ring, a vaginal sponge, and a vaginal applicator.


SEQUENCE LISTING (16 sequences)

SEQ ID NO: 1 - protein, 64 aa, Artificial Sequence
  consensus
GRXLXIPCRVTSPNXTVTLKKFPXDXLXPDGXRIXWDSRXGFIIXNATYKEIGLLXCEATVNGH

SEQ ID NO: 2 - protein, 64 aa, rat
GRELIIPCRVTSPNITVTLKKFPFDALTPDGQRIAWDSRRGFIIANATYKEIGLLTCEATVNGH

SEQ ID NO: 3 - protein, 64 aa, human
GRELVIPCRVTSPNITVTLKKFPLDTLIPDGKRIIWDSRKGFIISNATYKEIGLLTCEATVNGH

SEQ ID NO: 4 - protein, 64 aa, mouse
GRQLIIPCRVTSPNVTVTLKKFPFDTLTPDGQRITWDSRRGFIIANATYKEIGLLNCEATVNGH

SEQ ID NO: 5 - protein, 66 aa, Artificial Sequence
  consensus
GEKLVLNCTARTELNVGLDFXWXXPXSKXXHKKIVNRDXKXXXGXXXKXFLSTLTIXXVTXSDQGX

SEQ ID NO: 6 - protein, 66 aa, mouse
GEKLVLNCTARTELNVGLDFTWHSPPSKSHHKKIVNRDVKPFPGTVAKMFLSTLTIESVTKSDQGE

SEQ ID NO: 7 - protein, 66 aa, rat
GEKLVLNCTARTELNVGLDFSWQFPSSKHQHKKIVNRDVKSLPGTVAKMFLSTLTIDSVTKSDQGE

SEQ ID NO: 8 - protein, 76 aa, homo sapien
GEKLVLNCTARTELNVGIDFNWEYPSSKHQHKKLVNRDLKTQSGSEMKKFLSTLTIDGVTRSDQGLYTCAASSGLM

SEQ ID NO: 9 - protein, 222 aa, Artificial Sequence
  synthetic protein created in laboratory
TCPPCPAPELLGGPSVFLGPPKPKDTLMISRTPEVTCVVVDVSHEDPQVKFNWYVDGVXVHNAKTKPREXQYNSTYRVVSVLTVLHQXWLDGKEYKCKVSNKALPEPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 222 aa, Artificial Sequence
  synthetic protein created in laboratory
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGPFFLYSKLTVDKSRWQQGNVFSCSVMHEALHNHYTQKSLSLSPG

SEQ ID NO: 11 - protein, 222 aa, Artificial Sequence
  synthetic protein created in laboratory
TCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 6 aa, Artificial Sequence
  synthetic protein created in laboratory
AAAGGM

SEQ ID NO: 13 - protein, 14 aa, Artificial Sequence
  synthetic protein created in laboratory
AAAGGMPPAAAGGM

SEQ ID NO: 14 - protein, 6 aa, Artificial Sequence
  synthetic protein created in laboratory
AAAGGM

SEQ ID NO: 15 - protein, 9 aa, Artificial Sequence
  synthetic protein created in laboratory
PPAAAGGMM

SEQ ID NO: 16 - protein, 432 aa, Artificial Sequence
  synthetic protein created in laboratory
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGPFFLYSKLTVDKSRWQQGNVFSCSVMHEALHNHYTQKSLSLSPGK